(19) United States
(12) Patent Application Publication
(10) Pub. No.: US 2021/0208130 A1
(43) Pub. Date: Jul. 8, 2021

(54) ASSAYS FOR CELL-BASED THERAPIES OR TREATMENTS

(71) Applicant: The Regents of the University of California, Oakland, CA (US)

(72) Inventors: Jing YANG, Irvine, CA (US); Chinhui Gina HSIANG, Cerritos, CA (US); Henry KLASSEN, Irvine, CA (US)

(21) Appl. No.: 17/280,486

(22) PCT Filed: Sep. 27, 2019
(86) PCT No.: PCT/US19/53511

§ 371 Date: Mar. 26, 2021

Related U.S. Application Data
(60) Provisional application No. 62/737,359, filed on Sep. 27, 2018.

(51) Int. Cl. G01N 33/50; C12N 5/071; G01N 33/58

(52) U.S. Cl. CPC G01N 33/502; C12N 5/0602; C12N 2500/30; G01N 2800/52; G01N 2496/00; G01N 33/582

(57) ABSTRACT

The present disclosure provides in vitro methods for determining the potency of a cell-based therapy or treatment. In alternative embodiments, provided are compositions, including products of manufacture and kits, and methods, comprising (or comprising use of) quantitative in vitro assays for determining the potency of cell-based therapies or treatments, including those used in the treatment of retinal degeneration.

### RELATED APPLICATIONS

This application claims priority to, and the benefit of, U.S. Provisional Application No. 62/737,359, filed Sep. 27, 2018, the contents of which is incorporated herein by reference in its entirety.

### TECHNICAL FIELD

This invention generally relates to cell-based assays and therapies. In alternative embodiments, provided are compositions, including products of manufacture and kits, and methods, comprising (or comprising use of) quantitative in vitro assays for determining the potency of cell-based therapies or treatments, including those used in the treatment of retinal degeneration.

### BACKGROUND

Retinal degeneration refers to the deterioration or degeneration caused by the progressive and irreversible decline and death of photoreceptor cells in the retina. The death of photoreceptor cells can result in blindness. Stem cells and other pluripotent cells have been contemplated for use in treating patients with retinal degeneration and can be isolated from a number of sources including embryonic tissue, adult brain, genetically manipulated dermal fibroblasts and even the retina. However, testing these cell-based therapies and treatments, and more generally cell-based therapies and treatments directed towards a wide variety of diseases including cancer and autoimmune conditions, remains difficult as in vivo assays in humans or model organisms are costly, time-consuming and often lack quantitative results. Thus, there is a need in the art for a robust, cost-effective, time-efficient and quantitative in vitro assay for determining the potency of cell-based therapies or treatments, including those used in the treatment of retinal degeneration.

### SUMMARY

The present disclosure provides a method for measuring the potency of a cell-based therapy or treatment, the method comprising the steps of: incubating a first plurality of cells with a toxic compound and conditioned media, wherein the conditioned media comprises the media used to culture the cell-based therapy or treatment; incubating an at least second plurality of cells with the toxic compound and control media; determining the viability of the first plurality of cells and the at least second plurality of cells; and comparing the viability of the first plurality of cells with the viability of the second plurality of cells, thereby determining the potency of the cell-based therapy or treatment. The potency can be the ratio of the viability of the first plurality of cells with the viability of the second plurality of cells.

The present disclosure provides a method for measuring the potency of a cell-based therapy or treatment, the method comprising the steps of: incubating a first plurality of cells with a toxic compound and conditioned media, wherein the conditioned media comprises the media used to culture the cell-based therapy or treatment; incubating an at least second plurality of cells with the toxic compound and control media; determining the viability of the first plurality of cells and the at least second plurality of cells; determining the apoptosis activity in the first plurality of cells and the at least second plurality of cells; determining a fold change protection value of the first plurality of cells, wherein the fold change protection value is the ratio of viability of the first plurality of cells to the apoptosis activity in the first plurality of cells; determining a fold change protection value of the at least second plurality of cells, wherein the fold change protection value is the ratio of viability of the at least second plurality of cells to the apoptosis activity in the at least second plurality of cells; and determining the potency of the cell-based therapy or treatment, wherein the potency is the ratio of the fold change protection value of the first plurality of cells to the fold change protection value of the at least second plurality of cells.

The preceding methods can further comprise comparing the potency of the cell-based therapy or treatment to a predetermined cutoff value, wherein if the potency is greater than the predetermined cutoff value then the cell-based therapy or treatment is identified as sufficiently potent for administration to a subject.

The preceding methods can further comprise: comparing the potency of the cell-based therapy or treatment to a predetermined cutoff value; and administering to a subject in need thereof at least one therapeutically effective dose of the cell therapy or treatment when the potency is greater than the predetermined cutoff value.

A predetermined cutoff value can be about 2.

A cell-based therapy or treatment can comprise retinal progenitor cells (RPCs), retinal pigment epithelial cells (RPEs), ARPE-19 cells, neural stem/progenitor cells, mesenchymal stem cells, CD34+ cells, stem/progenitor cells, leukocytes, fibroblasts or any combination thereof. A cell-based therapy or treatment comprises RPCs.

A first plurality of cells and an at least second plurality of cells can comprise retinoblastoma (RB) cells, retinal pigment epithelial cells (RPEs), ARPE-19 cells, Müller cell-derived cells, MIO-M1 cells, neuronal cells, glial cells, fibroblasts, non-ocular cells or any combination thereof. A first plurality of cells and an at least second plurality of cells can comprise RB cells. A first plurality of cells and an at least second plurality of cells can comprise at least about 1,000 RB cells to at least about 250,000 RB cells in at least about 10 μl to at least about 40 μl of media. A first plurality of cells and an at least second plurality of cells can comprise at least about 25,000 RB cells in at least about 25 μl of media.

A first plurality of cells and an at least second plurality of cells can be incubated with at least about 50 μl to at least about 100 μl of conditioned media and control media, respectively. A first plurality of cells and an at least second plurality of cells can be incubated with at least about 75 μl of conditioned media and control media, respectively.

A toxic compound can induce apoptosis. A toxic compound can be sodium butyrate. Sodium butyrate can be present in a concentration of about 2 mM to about 32 mM. Sodium butyrate can be present in a concentration of about 16 mM.

A first plurality of cells and an at least second plurality of cells can be incubated for a time period of at least about 1 hour to at least about 72 hours. A first plurality of cells and an at least second plurality of cells can be incubated for a time period of at least about 46 hours.

Determining the viability of a first plurality of cells and an at least second plurality of cells can comprise measuring metabolic capacity of the first plurality of cells and the at least second plurality of cells. Metabolic capacity can be measured using a fluorescence-based assay.

A fluorescence-based assay can comprise: incubating the first plurality of cells and the at least second plurality of cells with resazurin (7-Hydroxy-3H-phenoxazin-3-one 10-oxide sodium salt) for at a period of at least about 1 hour; and measuring the fluorescence of the first plurality of cells and the at least second plurality of cells. A fluorescence-based assay can be a CellTiter-Blue® Cell Viability Assay. At least about 20 μl of 1:4 diluted CellTiter-Blue® reagent can be added to a first plurality of cells and to an at least second plurality of cells.

Apoptosis activity in a first plurality of cells and an at least second plurality of cells can be measured using a luminescence-based assay. A luminescence-based assay can comprise: incubating a first plurality of cells and an at least second plurality of cells with a luminogenic caspase-3/7 substrate for at least about 1 hours; and measuring the luminescence of the first plurality of cells and the at least second plurality of cells. A luminogenic caspase-3/7 substrate can comprise a tetrapeptide sequence DEVD that is cleaved by caspase-3 or caspase-7, thereby producing a luciferase substrate. A luminescence-based assay can be a Caspase-Glo® 3/7 assay system. At least about 120 μl of Caspase-Glo® 3/7 assay reagent can be added to the first plurality of cells and to the at least second plurality of cells.

The preceding methods can further comprise: incubating an at least third plurality of cells with a toxic compound and inactive conditioned media, wherein the inactive conditioned media comprises the media used to culture an inactive cell-based therapy or treatment; determining the viability of the at least third plurality of cells; determining the apoptosis activity in the at least third plurality of cells; and determining a fold change protection value of the at least third plurality of cells, wherein the fold change protection value is the ratio of viability to apoptosis activity; and determining the potency of the inactive cell-based therapy or treatment, wherein the potency is the ratio of the fold change protection value of the at least third plurality of cells to the fold change protection value of the at least second plurality of cells; and comparing the potency of the inactive cell-based therapy or treatment to a predetermined cutoff value, wherein if the potency of the inactive cell-based therapy is less than or equal to the predetermined cutoff value, then the method is identified as valid.

The preceding methods can further comprise: incubating an at least third plurality of cells with a toxic compound and inactive conditioned media, wherein the inactive conditioned media comprises the media used to culture an inactive cell-based therapy or treatment; determining the viability of the at least third plurality of cells; comparing the viability of the third plurality of cells with the viability of the second plurality of cells, thereby determining the potency of the inactive cell-based therapy or treatment; and comparing the potency of the inactive cell-based therapy or treatment to a predetermined cutoff value, wherein if the potency of the inactive cell-based therapy is less than or equal to the predetermined cutoff value, then the method is identified as valid.

An inactive cell-based therapy or treatment can comprise cutaneous T lymphocytes, HuT 78 cells or any combination thereof.

The preceding methods can further comprise: incubating an at least third plurality of cells with a toxic compound and active conditioned media, wherein the active conditioned media comprises the media used to culture an active cell-based therapy or treatment; determining the viability of the at least third plurality of cells; determining the apoptosis activity in the at least third plurality of cells; determining a fold change protection value of the at least third plurality of cells, wherein the fold change protection value is the ratio of viability to apoptosis activity; determining the potency of the active cell-based therapy or treatment, wherein the potency is the ratio of the fold change protection value of the at least third plurality of cells to the fold change protection value of the at least second plurality of cells, and comparing the potency of the active cell-based therapy or treatment to a predetermined cutoff value, wherein if the potency of the active cell-based therapy is greater than the predetermined cutoff value, then the method is identified as valid.

The preceding methods can further comprise: incubating an at least third plurality of cells with a toxic compound and active conditioned media, wherein the active conditioned media comprises the media used to culture an active cell-based therapy or treatment; determining the viability of the at least third plurality of cells; comparing the viability of the third plurality of cells with the viability of the second plurality of cells, thereby determining the potency of the active cell-based therapy or treatment; and comparing the potency of the active cell-based therapy or treatment to a predetermined cutoff value, wherein if the potency of the active cell-based therapy is greater than the predetermined cutoff value, then the method is identified as valid.

An active cell-based therapy can comprise retinal pigment epithelial cells (RPEs), ARPE-19 cells, fibroblasts, CCD-1112Sk cells or any combination thereof.

Control media can comprise standard media. A cell-based therapy or treatment is for treating a retinal disease or condition.

Provided are methods for measuring the potency or efficacy of a cell-based therapy or treatment, the methods comprising the steps of: 1) incubating a first plurality of cells with a candidate compound and the cell-based therapy or treatment; 2) incubating a second plurality of cells with a candidate compound; 3) determining the viability and/or metabolic activity of the first plurality of cells; 4) determining the viability and/or metabolic activity of the second plurality of cells; and 5) comparing the viability and/or metabolic activity of the first plurality of cells with the viability and/or metabolic activity of the second plurality of cells, thereby determining the potency of the treatment. In some aspects, the first plurality of cells is substantially the same as the second plurality of cells.

The first plurality of cells and the second plurality of cells can comprise the same cell type. By way of non-limiting example, the cell type can be human retinoblastoma cells.

The first plurality of cells and the second plurality of cells can each comprise between about 1,000 to about 250,000 cells (e.g., about 1,000; 25,000; 50,000; 75,000; 100,000; 125,000; 150,000; 175,000; 200,000; 225,000; or 250,000). In some non-limiting examples, the first plurality of cells and the second plurality of cells can each comprise about 25,000 cells.

The cell-based therapy or treatment can comprise conditioned medium.

The conditioned medium can be produced by collecting the medium used to culture a third plurality of cells. The third plurality of cells can comprise mammalian retinal progenitor cells. The mammalian retinal progenitor cells can be human retinal progenitor cells. The third plurality of cells can comprise between 0.1×10⁶ and 1×10⁷ human retinal progenitor cells (e.g., 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1×10⁶, 2×10⁶, 3×10⁶, 4×10⁶, 5×10⁶, 6×10⁶, 7×10⁶, 8×10⁶, 9×10⁶, or 1×10⁷ cells). In some non-limiting examples, the third plurality of cells can comprise about 9×10⁶ human retinal progenitor cells.

In other embodiments, the third plurality of cells comprises human retinal pigment epithelial cells (hRPEs) (see e.g., FIG. 8).

As used herein, the term “candidate compound” can refer to a toxic compound, a semi-toxic compound, or the like. By way of non-limiting example, the toxic compound can induce apoptosis. The toxic compound can be sodium butyrate. The sodium butyrate can be present in an amount between about 0 mM and about 26 mM (e.g., about 0, 0.1, 0.2, 0.3, 0.4, 0.5, 0.6, 0.7. 0.8, 0.9, 1, 2, 3, 4, 5, 6, 7, 8, 9, 10, 11, 12, 13, 14, 15, 16, 17, 18, 19, 20, 21, 22, 23, 24, 25, or 26 mM). In some non-limiting examples, the sodium butyrate can be present in an amount about 8 mM.

The first plurality of cells and the second plurality of cells can be incubated for a period of at least 1 hour, or at least 12 hours, or at least 24 hours, or at least 48 hours, or at least 72 or more hours. The first plurality of cells and the second plurality of cells can be incubated for about 2 hours.

Determining the viability of the first plurality and the second plurality of cells can comprise measuring the metabolic capacity of the first plurality of cells and the second plurality of cells. The metabolic capacity of the first plurality of cells and the second plurality of cells can be measured using a fluorescence-based assay.

The fluorescence-based assay can comprise: 1) incubating the first plurality of cells and the second plurality of cells with resazurin (7-Hydroxy-3H-phenoxazin-3-one 10-oxide sodium salt) for at a period of at least 1 hour; 2) measuring the fluorescence of the first plurality of cells and the second plurality of cells; and 3) comparing the measured fluorescence, thereby determining the viability of the first plurality of cells and the second plurality of cells.

Any of the above aspects can be combined with any other aspect.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The above and further features will be more clearly appreciated from the following detailed description when taken in conjunction with the accompanying drawings.

FIG. 1 illustrates a series of charts showing the results of a fluorescence-based cell viability assay used in the methods of the present disclosure.

FIG. 2 graphically illustrates a series of bar graphs showing the potency of human retinal progenitor cell conditioned medium (hRPC CM) at various concentrations of sodium butyrate as measured using the methods of the present disclosure. The blue or first bar in each group corresponds to cells incubated in standard medium (SM) and the orange or second bar in each group corresponds to cells incubated in hRPC CM.

FIG. 3 graphically illustrates a series of bar graphs showing the potency of hRPC CM at various concentrations of sodium butyrate as measured using the methods of the present disclosure. The blue or first bar in each group corresponds to cells incubated in SM and the orange or second bar in each group corresponds to cells incubated in hRPC CM.

FIG. 4 graphically illustrates a bar graph showing the potency of various dilutions of hRPC CM as measured using the methods of the present disclosure. The cyan or first bar in each group corresponds to cells incubated in SM. The grey or second bar in each group corresponds to cells incubated in hRPC CM with no dilution. The yellow or third bar in each group corresponds to cells incubated in hRPC CM diluted by a factor of 2. The dark blue or fourth bar in each group corresponds to cells incubated in hRPC CM diluted by a factor of 4. The green or fifth bar in each group corresponds to cells incubated in hRPC CM diluted by a factor of 8.

FIG. 5 graphically illustrates a bar graph showing the potency of hRPC CM produced using different amounts of human retinal progenitor cells (hRPCs) as measured using the methods of the present disclosure. The cyan or first bar in each group corresponds to cells incubated in SM. The orange or second bar in each group corresponds to cells incubated in hRPC CM produced using 9.0×10⁶ hRPCs. The grey or third bar in each group corresponds to cells incubated in hRPC CM produced using 6.0×10⁶ hRPCs.

FIG. 6 graphically illustrates a series of bar graphs showing the potency of hRPC CM produced from different populations of hRPCs as measured using the methods of the present disclosure. In the left panel, the cyan or first bar in each group corresponds to cells incubated with SM; the orange or second bar is cells incubated with hRPC CM produced from hRPCs from lot G1; the grey or third bar corresponds to cells incubated with hRPC CM produced from hRPCs from lot G2; the yellow or fourth bar in each group corresponds to cells incubated with hRPC CM produced using hRPCs from lot G3; and the dark blue or fifth bar in each group corresponds to cells incubated with hRPC CM produced using hRPCs from lot G5. In the right panel, the cyan or first bar in each group corresponds to cells incubated with SM; the orange or second bar is cells incubated with hRPC CM produced from hRPCs from lot G1; the grey or third bar corresponds to cells incubated with hRPC CM produced from hRPCs from lot G2; the yellow or fourth bar in each group corresponds to cells incubated with hRPC CM produced using hRPCs from lot G3; and the dark blue or fifth bar in each group corresponds to cells incubated with hRPC CM produced using hRPCs from lot G5.

FIG. 7 graphically illustrates a bar graph showing the potency of hRPC CM produced from different populations of hRPCs as measured using the methods of the present disclosure. The cyan or first bar in each group corresponds to cells incubated with SM; the orange or second bar is cells incubated with hRPC CM produced from hRPCs from lot G1; the grey or third bar corresponds to cells incubated with hRPC CM produced from hRPCs from lot G2; the yellow or fourth bar in each group corresponds to cells incubated with hRPC CM produced using hRPCs from lot G3; the dark blue or fifth bar in each group corresponds to cells incubated with hRPC CM produced using hRPCs from lot G4; the green or sixth bar in each group corresponds to cells incubated with hRPC CM produced using hRPCs from lot G5; the light blue or seventh bar in each group corresponds to cells incubated with hRPC CM produced using hRPCs from lot L-SB; and the pink or eighth bar in each group corresponds to cells incubated with hRPC CM produced using hRPCs from lot L-PB.

FIG. 8 graphically illustrates a series of bar graphs showing the potency of conditioned media produced using various cells types as measured using the methods of the present disclosure. In the left panel, the blue or first bar in each group corresponds to cells incubated in SM; the orange or second bar in each group corresponds to cells incubated in hRPC CM; and the yellow or third bar in each group corresponds to cells incubated in conditioned medium produced using human retinoblastoma cells. In the right panel, the cyan or first bar in each group corresponds to cells incubated in SM and the green or second bar in each group corresponds to cells incubated in conditioned medium produced using human retinal pigment epithelial cells.

FIG. 9 shows gene expression data for selected cytokines, chosen as candidate neurotrophic factors for hRPCs. Data was obtained via qPCR, from multiple cell types, including (from left to right) hRPC, hRB, hRPE, and hFB. Retinal cell types group separately from fibroblasts. The hRB line was derived from tumor, all other cell types from fetal tissue. Expression of putative trophic factor OPN can be seen to be highest for hRPCs.

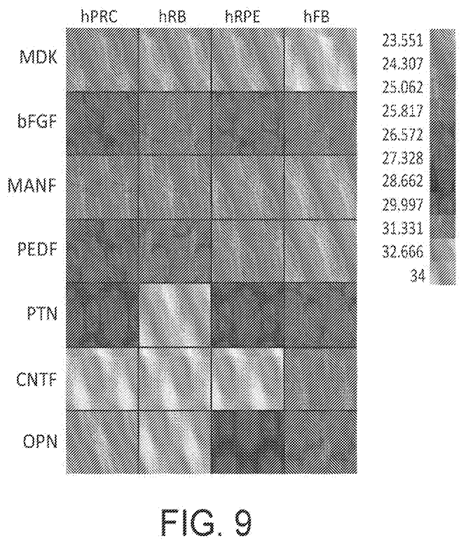

FIG. 10 graphically illustrates a series of graphs showing the results from a multiplexed potency assay of the present disclosure. The top left panel shows the measured cell viability. The top right panel shows the measured apoptotic activity. The bottom panel shows calculated potency values using the data shown in the top two panels.

FIG. 11 graphically illustrates the results of a multiplexed potency assay of the present disclosure testing unfiltered and filtered conditioned media.

FIG. 12 graphically illustrates the results of a multiplexed potency assay of the present disclosure testing negative control conditioned media and positive control conditioned media.

FIG. 13 graphically illustrates the results of a multiplexed potency assay of the present disclosure testing conditioned media derived from various lots of hRPCs.

FIG. 14 graphically illustrates the results of a multiplexed potency assay of the present disclosure testing conditioned media derived from hRPC cultures with different seeding densities.

FIG. 15 graphically illustrates the results of a multiplexed potency assay of the present disclosure testing various dilutions of conditioned media, indicating that the multiplexed potency assay exhibits linearity of response.

### DETAILED DESCRIPTION

The most common type of inherited retinal disease (dystrophy) is retinitis pigmentosa (RP) and the most common degenerative disease is age-related macular degeneration (AMD). Potential therapies include the use of human retinal progenitor cells (hRPC) in the treatment of RP and the use of stem cell-derived human pigment epithelial (RPE) cell products in the treatment of AMD. Tests of hRPCs in the treatment of RP have indicated that the treatment relies on a neurotrophic effect that is likely to result from a cocktail of multiple cytokines. The role of these cytokines has been only partially delineated at present. Because the potency of a manufactured hRPC cell product might be expected to vary from batch to batch, it would be helpful to have a simple and convenient in vitro potency assay capable of prospectively measuring the trophic efficacy of a given lot of manufactured cell product prior to use in patients, without the need for in vivo testing and related infrastructure and personnel requirements.

The current method of evaluating hRPC treatment potency is in vivo testing in Royal College of Surgeons (RCS) rats, which are a well-characterized model of autosomal recessive RP. To perform these tests, a vivarium with a RCS colony is required, along with a surgical suite, electrophysiology suite, and ocular histology lab, all of which must be staffed with associated personnel with specialized expertise in these areas. Thus, the current in vivo testing method is cumbersome, skill intensive, labor intensive, time intensive, and resource intensive.

The neurotrophic effect of the hRPC treatment has rendered it difficult to recapitulate in an in vitro system. For instance, the trophic effect of the treatment observed in vivo seems to occur at the level of photoreceptors, which are cells that challenging to maintain in vitro. In one aspect, the methods of the present disclosure provide a means of measuring the potency of a manufactured cell product, without the need for testing in animals and prior to use in humans. The methods of the present disclosure may only require the use of cell culture facilities, a modest array of equipment, plus the labor of one experienced technician over the course of a single day. Also needed is a readily available immortal cell line or a human tumor cell line and a number of other readily available reagents. The test article is a small sample of cell culture media previously taken from living culture of a specific therapeutic cell type. Note that the test facility need not deal with the handling a delicate cell type. Thus, the methods of the present disclosure, as compared with existing in vivo assays, are inexpensive, require minimum labor and are time effective. The methods of the present disclosure reduce what was previously a multi-month in vivo process requiring a specialized team to a one day, one technician in vitro process.

The present disclosure provides methods that measure the potency of a cell-based therapy or treatment. The methods provide a quantitative in vitro potency assay that can be used to determine how potently a cell-based therapy or treatment increases the viability of a population of cells. Thus, the assay provides a method of quantifying the potency of a trophic effect of a cell-based therapy or treatment. In one aspect, the methods of the present disclosure use a toxic compound to provide a metabolic insult to a population of cells to better detect the potency of the trophic effect of a cell-based therapy or treatment. In one non-limiting example, the methods of the present disclosure can be used to test the potency of the trophic effect of donor fetal retinal cells (retinal progenitor cells) on a host retina, notably including host cones. Donor fetal retinal cells have been shown to have a trophic effect that is not only neuroprotective but also has a rapid revitalizing effect on residual host retinal cells as determined by improved visual function. Donor cells are capable of integrating into the retina and, via cellular differentiation, replace photoreceptors (which can be in limited numbers). The overall effect is to both rapidly and sustainably restore and preserve clinically significant degrees of visual function in a retina otherwise destined to fail completely, leaving the patient completely blind.

The present disclosure provides an in vitro method for measuring the potency of a cell-based therapy or treatment, the method comprising the steps of: incubating a first plurality of cells with a toxic compound and the cell-based therapy or treatment; incubating a second plurality of cells with a toxic compound; determining the viability of the first plurality of cells; determining the viability of the second plurality of cells; and comparing the viability of the first plurality of cells with the viability of the second plurality of cells, thereby determining the potency of the treatment.

The present disclosure also provides a method for measuring the potency of a cell-based therapy or treatment, the method comprising the steps of: incubating a first plurality of cells with a toxic compound and conditioned media, wherein the conditioned media comprises the media used to culture the cell-based therapy or treatment; incubating an at least second plurality of cells with the toxic compound and control media; determining the viability of the first plurality of cells and the at least second plurality of cells; comparing the viability of the first plurality of cells with the viability of the second plurality of cells, thereby determining the potency of the cell-based therapy or treatment.

In some aspects of the preceding methods, wherein the potency is the ratio of the viability of the first plurality of cells with the viability of the second plurality of cells.

In some aspects, the preceding methods can further comprise incubating an at least third plurality of cells with a toxic compound and inactive conditioned media, wherein the inactive conditioned media comprises the media used to culture an inactive cell-based therapy or treatment; determining the viability of the at least third plurality of cells; comparing the viability of the third plurality of cells with the viability of the second plurality of cells, thereby determining the potency of the inactive cell-based therapy or treatment, comparing the potency of the inactive cell-based therapy or treatment to a predetermined cutoff value, wherein if the potency of the inactive cell-based therapy is less than or equal to the predetermined cutoff value, then the method is identified as valid. Without wishing to be bound by theory, an inactive conditioned media that comprises the media used to culture an inactive cell-based therapy or treatment is a conditioned media that is known to be non-active, will not protect the third plurality of cells from the deleterious effects of the toxic compound (e.g. sodium butyrate), and therefore should not have a potency value above a certain predetermined value. Thus, the inactive conditioned media serves as a negative control—if the results of a particular assay indicate that the inactive conditioned media has low to no potency, the results of the assay can be viewed as more accurate. However, if the results of a particular assay indicate that the inactive conditioned media has potency, then the assay results may be compromised and not reflective of the actual potency of the cell-based treatments or therapies tested. The inactive cell-based therapy or treatment can comprise cutaneous T lymphocytes, HuT 78 cells or any combination thereof.

In some aspects, the preceding methods can further comprise incubating an at least third plurality of cells with a toxic compound and active conditioned media, wherein the active conditioned media comprises the media used to culture an active cell-based therapy or treatment; determining the viability of the at least third plurality of cells; comparing the viability of the third plurality of cells with the viability of the second plurality of cells, thereby determining the potency of the active cell-based therapy or treatment, comparing the potency of the active cell-based therapy or treatment to a predetermined cutoff value, wherein if the potency of the active cell-based therapy is greater than the predetermined cutoff value, then the method is identified as valid. Without wishing to be bound by theory, an active conditioned media that comprises the media used to culture an active cell-based therapy or treatment is a conditioned media that is known to be active, will protect the third plurality of cells from the deleterious effects of the toxic compound (e.g. sodium butyrate), and therefore should have a potency value above a certain predetermined value. Thus, the active conditioned media serves as a positive control—if the results of a particular assay indicate that the active conditioned media has low to no potency, then the assay results may be compromised and not reflective of the actual potency of the cell-based treatments or therapies tested. However, if the results of a particular assay indicate that the active conditioned media has potency, then the results may be viewed as accurate. The active cell-based therapy or treatment can comprise retinal pigment epithelial cells (RPEs), ARPE-19 cells, fibroblasts, CCD-1112Sk cells or any combination thereof.

The present disclosure also provides a method for measuring the potency of a cell-based therapy or treatment, the method comprising the steps of: incubating a first plurality of cells with a toxic compound and the cell-based therapy or treatment; incubating an at least second plurality of cells with the toxic compound and control media; determining the viability of the first plurality of cells and the at least second plurality of cells; determining the apoptosis activity in the first plurality of cells and the at least second plurality of cells; and determining a fold change protection value of the first plurality of cells, wherein the fold change protection value is the ratio of viability of the first plurality of cells to the apoptosis activity in the first plurality of cells; determining a fold change protection value of the at least second plurality of cells, wherein the fold change protection value is the ratio of viability of the at least second plurality of cells to the apoptosis activity in the at least second plurality of cells; and determining the potency of the cell-based therapy or treatment, wherein the potency is the ratio of the fold change protection value of the first plurality of cells to the fold change protection value of the at least second plurality of cells. As used herein, this method is referred to as a “multiplexed potency assay”.

The present disclosure also provides a method for measuring the potency of a cell-based therapy or treatment, the method comprising the steps of: incubating a first plurality of cells with a toxic compound and conditioned media, wherein the conditioned media comprises the media used to culture the cell-based therapy or treatment; incubating an at least second plurality of cells with the toxic compound and control media; determining the viability of the first plurality of cells and the at least second plurality of cells; determining the apoptosis activity in the first plurality of cells and the at least second plurality of cells; and determining a fold change protection value of the first plurality of cells, wherein the fold change protection value is the ratio of viability of the first plurality of cells to the apoptosis activity in the first plurality of cells; determining a fold change protection value of the at least second plurality of cells, wherein the fold change protection value is the ratio of viability of the at least second plurality of cells to the apoptosis activity in the at least second plurality of cells; and determining the potency of the cell-based therapy or treatment, wherein the potency is the ratio of the fold change protection value of the first plurality of cells to the fold change protection value of the at least second plurality of cells. As used herein, this method is also referred to as a “multiplexed potency assay”.

The preceding methods can further comprise comparing the potency of the cell-based therapy or treatment to a predetermined cutoff value, wherein if the potency is greater than the predetermined cutoff value then the cell-based therapy or treatment is identified as sufficiently potent for administration to a subject.

The preceding methods can further comprise comparing the potency of the cell-based therapy or treatment to a predetermined cutoff value; and administering to a subject in need thereof at least one therapeutically effective dose of the cell therapy or treatment when the potency is greater than the predetermined cutoff value.

The preceding methods can further comprise incubating an at least third plurality of cells with a toxic compound and inactive conditioned media, wherein the inactive conditioned media comprises the media used to culture an inactive cell-based therapy or treatment; determining the viability of the at least third plurality of cells; determining the apoptosis activity in the at least third plurality of cells; and determining a fold change protection value of the at least third plurality of cells, wherein the fold change protection value is the ratio of viability to apoptosis activity; and determining the potency of the inactive cell-based therapy or treatment, wherein the potency is the ratio of the fold change protection value of the at least third plurality of cells to the fold change protection value of the at least second plurality of cells; comparing the potency of the inactive cell-based therapy or treatment to a predetermined cutoff value, wherein if the potency of the inactive cell-based therapy is less than or equal to the predetermined cutoff value, then the method is identified as valid. Without wishing to be bound by theory, an inactive conditioned media that comprises the media used to culture an inactive cell-based therapy or treatment is a conditioned media that is known to be non-active, will not protect the third plurality of cells from the deleterious effects of the toxic compound (e.g. sodium butyrate), and therefore should not have a potency value above a certain predetermined value. Thus, the inactive conditioned media serves as a negative control—if the results of a particular assay indicate that the inactive conditioned media has low to no potency, the results of the assay can be viewed as more accurate. However, if the results of a particular assay indicate that the inactive conditioned media has potency, then the assay results may be compromised and not reflective of the actual potency of the cell-based treatments or therapies tested. The inactive cell-based therapy or treatment can comprise cutaneous T lymphocytes, HuT 78 cells or any combination thereof.

In some aspects, the preceding methods can comprise incubating an at least third plurality of cells with a toxic compound and active conditioned media, wherein the active conditioned media comprises the media used to culture an active cell-based therapy or treatment; determining the viability of the at least third plurality of cells; determining the apoptosis activity in the at least third plurality of cells; determining a fold change protection value of the at least third plurality of cells, wherein the fold change protection value is the ratio of viability to apoptosis activity; determining the potency of the active cell-based therapy or treatment, wherein the potency is the ratio of the fold change protection value of the at least third plurality of cells to the fold change protection value of the at least second plurality of cells; and comparing the potency of the active cell-based therapy or treatment to a predetermined cutoff value, wherein if the potency of the active cell-based therapy is greater than the predetermined cutoff value, then the method is identified as valid. Without wishing to be bound by theory, an active conditioned media that comprises the media used to culture an active cell-based therapy or treatment is a conditioned media that is known to be active, will protect the third plurality of cells from the deleterious effects of the toxic compound (e.g. sodium butyrate), and therefore should have a potency value above a certain predetermined value. Thus, the active conditioned media serves as a positive control—if the results of a particular assay indicate that the active conditioned media has low to no potency, then the assay results may be compromised and not reflective of the actual potency of the cell-based treatments or therapies tested. However, if the results of a particular assay indicate that the active conditioned media has potency, then the results may be viewed as accurate. The active cell-based therapy or treatment can comprise retinal pigment epithelial cells (RPEs), ARPE-19 cells, fibroblasts, CCD-1112Sk cells or any combination thereof.

In some aspects, the predetermined cutoff value can be about 0.5, or about 1.0, or about 1.5, or about 2.0, or about 3.0, or about 3.5, or about 4.0, or about 4.5, or about 5.0, or about 5.5, or about 6.0, or about 6.5, or about 7.0, or about 7.5, or about 8.0, or about 8.5, or about 9.0, or about 9.5, or about 10, or about 15, or about 20, or about 25, or about 30, or about 35, or about 40, or about 45, or about 50, or about 60, or about 70, or about 80, or about 90, or about 100.

In some aspects of methods of the present disclosure, a cell-based therapy or treatment comprises conditioned medium. “Conditioned medium” refers to a medium that is altered as compared to a standard, base or basal medium. The conditioning of a medium may cause molecules, such as nutrients and/or growth factors, to be added to or depleted from the original levels found in the base medium. In some aspects, a medium is conditioned by allowing cells of certain types to be grown or maintained in the medium under certain conditions for a certain period of time. In some aspects of the present disclosure, a conditioned medium is produced by collecting the medium used to culture a third plurality of cells. In a non-limiting example, the third plurality of cells can comprise mammalian retinal progenitor cells (RPCs), human retinal progenitor cells (hRPCs), mammalian retinal pigment epithelial cells (RPEs), human retinal pigment epithelial cells (hRPEs), ARPE-19 cells, neural stem/progenitor cells, mesenchymal stem cells, CD34+ cells, stem/progenitor cells, leukocytes, fibroblasts or any combination thereof. The mammalian retinal progenitor cells can be human retinal progenitor cells (hRPCs). The third plurality of cells can comprise hRPCs. In some aspects, a medium can be conditioned by allowing retinal progenitor cells to be expanded, differentiated or maintained in a medium of defined composition at a defined temperature for a defined number of hours. As will be appreciated by those of skill in the art, numerous combinations of cells, media types, durations and environmental conditions can be used to produce nearly an infinite array of conditioned media.

In some aspects of the methods as provided herein, conditioned medium can be produced by collecting the medium used to culture, expand, differentiate or maintain a third plurality of cells comprising between 1×10⁶ and 1×10⁷ human retinal progenitor cells. In other aspects, the third plurality of cells can comprise about 9×10⁶ human retinal progenitor cells. In some aspects, the third plurality of cells can comprise about 8×10⁶ human retinal progenitor cells. In some aspects, the third plurality of cells can comprise about 6×10⁶ human retinal progenitor cells. In some aspects, the third plurality of cells can comprise about 4×10⁶ human retinal progenitor cells. Methods of culturing and producing conditioned medium from human retinal progenitor cells are described in WO 2012/158910, which is herein incorporated by reference in its entirety. In some aspects, the conditioned medium can be produced by collecting the medium used to culture, expand differentiate or maintain a third plurality of cells that were seeded at a density of at least about 1×10⁶ cells, or at least about 2×10⁶ cells, or at least about 3×10⁶ cells, or at least about 4×10⁶ cells, or at least about 5×10⁶ cells, or at least about 6×10⁶ cells, or at least about 7×10⁶ cells, or at least about 8×10⁶ cells, or at least about 9×10⁶ cells, or at least about 10×10⁶ cells. In some aspects, the conditioned medium can be produced by collecting the medium used to culture, expand differentiate or maintain a third plurality of cells that were cultured for at least about 4 hours, or at least about 8 hours, or at least about 12 hours, or at least about 16 hours, or at least about 20 hours, or at least about 24 hours, or at least about 36 hours, or at least about 48 hours, or at least about 60 hours, or at least about 72 hours following seeding.

In some aspects of the methods of the present disclosure, conditioned media can be filtered prior to use in an assay of the present disclosure. In some aspects, conditioned media can be filtered using a concentrator or filter device in combination with centrifugation. In some aspects, the conditioned media can be filtered through a filter with a MWCO of at least about 3 kDa, or at least about 10 kDa, or at least about 30 kDa, or at least about 50 kDA, or at least about 100 kDa. In some aspects, after conditioned media is filtered, the “retentate” or the “top” fraction can be isolated for use in a method of the present disclosure. In some aspects, after conditioned media is filtered, either the “filtrate” or the “bottom” fraction can be isolated for use in a method of the present disclosure.

In some aspects of the methods as provided herein, the cell-based therapy or treatment comprises mammalian retinal progenitor cells (RPCs), human retinal progenitor cells (hRPCs), mammalian retinal pigment epithelial cells (RPEs), human retinal pigment epithelial cells (hRPEs), ARPE-19 cells, neural stem/progenitor cells, mesenchymal stem cells, CD34+ cells, stem/progenitor cells, leukocytes, fibroblasts or any combination thereof. The mammalian retinal progenitor cells can be human retinal progenitor cells (hRPCs). The cell-based therapy or treatment can comprise hRPCs. The cells may be genetically modified cells. In a non-limiting example, the genetically modified cells can have been transfected with a gene to express at least one polypeptide. In a non-limiting example, the genetically modified cells can have been infected by contacting cells with at least one viral particle, wherein the viral particle comprises at least one polynucleotide.

In some aspects of the methods as provided herein, a cell-based therapy or treatment or a conditioned media can comprise exosomes and/or microvesicles. A cell-based therapy or treatment or a conditioned media can comprise a fraction that is enriched in exosomes and/or microvesicles. The exosomes and/or microvesicles can be derived from any cell type, including, but not limited to mammalian retinal progenitor cells (RPCs), human retinal progenitor cells (hRPCs), mammalian retinal pigment epithelial cells (RPEs), human retinal pigment epithelial cells (hRPEs), ARPE-19 cells, neural stem/progenitor cells, mesenchymal stem cells, CD34+ cells, stem/progenitor cells, leukocytes, fibroblasts or any combination thereof. The cells may be genetically modified cells. In a non-limiting example, the genetically modified cells can have been transfected with at least one gene to express one polypeptide. In a non-limiting example, the genetically modified cells can have been infected by contacting cells with at least one viral particle, wherein the viral particle comprises at least one polynucleotide. The exosomes and/or microvesicles, or the fraction enriched in exosomes and/or microvesicles can be purified using exosomes/microvesicle techniques standard in the art, including, but not limited to, centrifugation, ultracentrifugation, size exclusion chromatography, ion exchange chromatography, immunoaffinity chromatography, or any other techniques standard in the art.

In some aspects of the methods of the present disclosure, control media comprises standard media.

In some aspects of the methods of the present disclosure, a cell-based therapy or treatment can be for treating a retinal disease or condition in a subject in need thereof. A retinal disease or condition can include, but is not limited to, Usher's disease, retinitis pigmentosa (RP), a degenerative retinal disease, an age related macular degeneration (AMD), a wet AMD or a dry AMD, geographic atrophy, a retinal photoreceptor disease, a diabetic retinopathy, cystoid macular edema, uveitis, a retinal detachment, a retinal injury, macular holes, macular telangiectasia, a traumatic or an iatrogenic retinal injury, a ganglion cell or optic nerve cell disease, a glaucoma or an optic neuropathy, an ischemic retinal disease such as retinopathy of prematurity, retinal vascular occlusion, or ischemic optic neuropathy; or improving a photopic (day light) vision; or for improving correcting visual acuity, or improving macular function, or improving a visual field, or improving scotopic (night) vision.

In some aspects of the preceding method, a first plurality of cells, a second plurality of cells, a third plurality of cells or any combination thereof can comprise the same cell type or can be different cell types. A first plurality of cells, a second plurality of cells, a third plurality of cells or any combination thereof can comprise a mixed population of different cell types. The cell type can be any primary cells isolated from a biological sample and/or non-immortal cell type cells (i.e., cells differentiated from pluripotent/stem cell populations). The cell type can be any immortalized cell line, including, but not limited to spontaneously immortalized cells. The cell type can be mammalian retinoblastoma cells (RBs), mammalian retinal pigment epithelial cells (RPEs), mammalian retinal progenitor cells (RPCs), ARPE-19 cells, Müller cell-derived cells, MIO-M1 cells, neuronal cells, glial cells, fibroblasts, non-ocular cells or any combination thereof. The cell type can be human retinoblastoma cells (hRBs), human retinal pigment epithelial cells or human retinal progenitor cells. A first plurality of cells, a second plurality of cells, a third plurality of cells or any combination thereof can be grown in suspension during cell culture. A first plurality of cells, a second plurality of cells, a third plurality of cells or any combination thereof can be grown using adherent cell culture methods.

In some aspects of methods of the present disclosure, the first plurality of cells and the second plurality of cells can each comprise between about 1,000 to about 250,000 cells. By way of non-limiting examples, the first plurality of cells and the second plurality of cells can each comprise about 25,000 cells. In some aspects of the methods of the present disclosure, the number of cells in the first plurality, the second plurality of cells, the third plurality of cells or any combination thereof can be scaled according to the size of microtiter plate that is being used for the assay. In a non-limiting example, the first plurality of cells, the second plurality of cells, the third plurality of cells or any combination thereof can comprise at least about 25,000 cells when the methods of the present disclosure are performed in a 96 well plate. When a 6 well plate is used, the first plurality of cells, the second plurality of cells, the third plurality of cells or any combination thereof can comprise at least about 400,000 cells. When a 12 well plate is used, the first plurality of cells, the second plurality of cells, the third plurality of cells or any combination thereof can comprise at least about 200,000 cells. When a 24 well plate is used, the first plurality of cells, the second plurality of cells, the third plurality of cells or any combination thereof can comprise at least about 100,000 cells. When a 48 well plate is used, the first plurality of cells, the second plurality of cells, the third plurality of cells or any combination thereof can comprise at least about 50,000 cells. When a 384 well plate is used, the first plurality of cells, the second plurality of cells, the third plurality of cells or any combination thereof can comprise at least about 6,250 cells. When a 1536 well plate is used, the first plurality of cells, the second plurality of cells, the third plurality of cells or any combination thereof can comprise at least about 1,562 cells.

In some aspects of the methods of the present disclosure, prior to the addition of a toxic compound and conditioned and/or standard media, the first plurality of cells, the second plurality of cells, the third plurality of cells or any combination thereof can be suspended in at least about 25 μl of standard media. The amount of standard media can used can be adjusted based on the size of microtiter plate that is being used for the assay. In a non-limiting example, when a 6 well plate is used, about 400 μl of standard media can be used. In a non-limiting example, when a 12 well plate is used, about 200 μl of standard media can be used. In a non-limiting example, when a 24 well plate is used, about 100 μl of standard media can be used. In a non-limiting example, when a 48 well plate is used, about 50 μl of standard media can be used. In a non-limiting example, when a 96 well plate is used, about 25 μl of standard media can be used. In a non-limiting example, when a 384 well plate is used, about 6.25 μl of standard media can be used. In a non-limiting example, when a 1536 well plate is used, about 1.56 μl of standard media can be used.

In some aspects of the methods of the present disclosure, the first plurality of cells, the second plurality of cells, the third plurality of cells or any combination thereof can be incubated with at least about 50 μl to at least about 100 μl of conditioned media or control media. In some aspects, the first plurality of cells, the second plurality of cells, the third plurality of cells or any combination thereof can be incubated with at least about 75 μl of conditioned media or control media. The amount of conditioned media or control media can be adjusted based on the size of microtiter plate that is being used for the assay. In a non-limiting example, when a 6 well plate is used, about 1200 μl of conditioned media or control media can be used. In a non-limiting example, when a 12 well plate is used, about 600 μl of conditioned media or control media can be used. In a non-limiting example, when a 24 well plate is used, about 300 μl of conditioned media or control media can be used. In a non-limiting example, when a 48 well plate is used, about 150 μl of conditioned media or control media can be used. In a non-limiting example, when a 96 well plate is used, about 75 μl of conditioned media or control media can be used. In a non-limiting example, when a 384 well plate is used, about 18.75 μl of conditioned media or control media can be used. In a non-limiting example, when a 1536 well plate is used, about 4.68 μl of conditioned media or control media can be used.

In some aspects of the methods as provided herein the toxic compound can induce apoptosis. In some aspects, the toxic compound can induce apoptosis, autophagy, Type I cell-death, Type II cell-death, necrosis, necroptosis, macroautophagy, anoikis, cornification, excitotoxicity, ferroptosis, activation-induced cell death, ischemic cell death, oncosis, pyroptosis, or any combination therefore.

Toxic compounds can include, but are not limited to, alkylating agents, antimetabolites, antitumor antibiotic, chemotherapeutic agents, alkaloids, taxanes, anti-microtubule agents, toxins, membrane permeabilizers, enzyme inhibitors, antimetabolites, mitotic inhibitors, DNA-repair enzyme inhibitors, DNA-damaging agents, UV radiation, gamma radiation, busulfan, cytosine, etoposide, bleomycin, 1-asparaginase, carmustine, arabinoside, teniposide, dactinomycin, hydroxyurea, chlorambucil, floxuridine, vinblastine, daunorubicin, procarbazine, cisplatin, fluorouracil, vincristine, doxorubicin, cyclophosphamide, mercaptopurine, vindesine, mitomycin-c, ifosfamide, methotrexate, taxoids, mitoxantrone, melphalan, gemcitabine, plicamycin, pemetrexed anthracyclines, and/or epothilones.

In some aspects of the methods of the present disclosure, the toxic compound is sodium butyrate. The sodium butyrate can be present in a concentration between about 1 mM and about 26 mM. The sodium butyrate can be present in a concentration between about 2 mM and about 24 mM. The sodium butyrate can be present in a concentration between about 0 mM and about 32 mM. In some aspects of the methods of the present disclosure, the sodium butyrate is present in a concentration of about 0 mM, i.e. there is no sodium butyrate added. In some aspects of the methods of the present disclosure the sodium butyrate is present in a concentration of about 2 mM. In some aspects of the methods of the present disclosure the sodium butyrate is present in a concentration of about 4 mM. In some aspects of the methods of the present disclosure the sodium butyrate is present in a concentration of about 6 mM. In some aspects of the methods of the present disclosure, the sodium butyrate is present in a concentration of about 8 mM. In some aspects of the methods of the present disclosure, the sodium butyrate is present in a concentration of about 10 mM. In some aspects of the methods of the present disclosure, the sodium butyrate is present in a concentration of about 12 mM. In some aspects of the methods of the present disclosure, the sodium butyrate is present in a concentration of about 14 mM. In some aspects of the methods of the present disclosure, the sodium butyrate is present in a concentration of about 16 mM. In some aspects of the methods of the present disclosure, the sodium butyrate is present in a concentration of about 18 mM, or about 20 mM, or about 22 mM, or about 24 mM, or about 26 mM, or about 28 mM, or about 30 mM, or about 32 mM, or about 34 mM, or about 36 mM, or about 38 mM, or about 40 mM.

In some aspects of the methods of the present disclosure, the first plurality of cells, the second plurality of cells, the third plurality of cells or any combination thereof are incubated for a period of at least 1 hour, or at least 12 hours, or at least 24 hours, or at least 46 hours, or at least 48 hours, or at least 72 hours or more prior to determining the viability of the cells. In some aspects, the first plurality of cells, the second plurality of cells, the third plurality of cells or any combination thereof are incubated for about 1 hour, or about 2 hours, or about 72 hours or more. In some aspects, the first plurality of cells, the second plurality of cells, the third plurality of cells or any combination thereof incubated for at least 46 hours prior to determining the viability of the cells.

In some aspects of the methods as provided herein, determining the viability of the first plurality of cells, the second plurality of cells, the third plurality of cells or any combination thereof can comprise using any cell viability assays known in the art. These include, but are not limited to, an ATP test assay, a Calcein AM assay, a Clonogenic assay, an ethidium homodimer assay, an Evans blue assay, a fluorescein diacetate hydrolysis/propidium iodide staining assay, a flow cytometry assay, a Formazan-based assay, an MTT assay, an XTT assay, green fluorescent protein assay, a lactate dehydrogenase assay, a methyl violet assay, a propidium iodide assay, a resazurin assay, a trypan blue assay, a terminal deoxynucleotidyl transferase dUTP nick end labeling (TUNEL) assay, or a combination thereof.

In some aspects of the methods of the present disclosure, determining the viability of the first plurality of cells, the second plurality of cells, the third plurality of cells or any combination thereof can comprise determining the proliferation of the first plurality of cells, the second plurality of cells, the third plurality of cells or any combination thereof. In some aspects of the methods of the present disclosure, determining the viability of the first plurality of cells, the second plurality of cells, the third plurality of cells or any combination thereof comprises measuring the metabolic capacity of the first plurality of cells, the second plurality of cells, the third plurality of cells or any combination thereof.

The metabolic capacity of the first plurality of cells, the second plurality of cells, the third plurality of cells or any combination thereof can be measured using a fluorescence-based assay. A fluorescence-based assay can comprise: 1) incubating the first plurality of cells, the second plurality of cells, the third plurality of cells or any combination thereof with resazurin (7-Hydroxy-3H-phenoxazin-3-one 10-oxide sodium salt) for at a period of at least 1 hour, 2) measuring the fluorescence of the first plurality of cells, the second plurality of cells, the third plurality of cells or any combination thereof. The fluorescence-based assay can further comprise 3) comparing the measured fluorescence, thereby determining the viability of the first plurality of cells, the second plurality of cells, the third plurality of cells or any combination thereof. In some aspects, the cells are incubated with resazurin for a time period of at least about 1 hour, or at least about 1.5 hours, or at least about 2.0 hours, or at least about 2.5 hours, or at least about 3.0 hours, or at least about 3.5 hours, or at least about 4.0 hours, or at least about 4.5 hours, or at least about 5.0 hours, or at least about 5.5 hours, or at least about 6.0 hours, or at least about 6.5 hours, or at least about 7.0 hours, or at least about 7.5 hours, or at least about 8.0 hours, or at least about 8.5 hours, or at least about 9.0 hours, or at least about 9.5 hours, or at least about 10 hours, or at least about 15 hours, or at least about 20 hours. The metabolic capacity of the first plurality of cells, the second plurality of cells, the third plurality of cells or any combination thereof can be measured using the CellTiter-Blue® Cell Viability Assay. In some aspects, the CellTiter-Blue® reagent can be diluted by 1:4. In some aspects, the CellTiter-Blue® reagent can be diluted by 1:4 in Dulbecco's PBS. In some aspects the CellTiter-Blue® reagent is undiluted. In some aspects, the first plurality of cells, the second plurality of cells, the third plurality of cells or any combination thereof can be incubated with at least about 20 μl of undiluted or diluted CellTiter-Blue® reagent. In some aspects, the amount of undiluted or diluted CellTiter-Blue® reagent used can be adjusted based on the microtiter plate that is being used for the assay.

In some aspects of the methods of the present disclosure determining the apoptosis activity in the first plurality of cells, the second plurality of cells, the third plurality of cells or any combination thereof can comprise a luminescence-based assay. The luminescence-based assay can comprise: incubating the first plurality of cells, the second plurality of cells, the third plurality of cells or any combination thereof with a luminogenic caspase-3/7 substrate for at least about 1 hours; and measuring the luminescence of the first plurality of cells, the second plurality of cells, the third plurality of cells or any combination thereof. The incubation with the luminogenic caspase-3/7 substrate can be for a time period of at least about 1 hour, or at least about 1.5 hours, or at least about 2.0 hours, or at least about 2.5 hours, or at least about 3.0 hours, or at least about 3.5 hours, or at least about 4.0 hours, or at least about 4.5 hours, or at least about 5.0 hours, or at least about 5.5 hours, or at least about 6.0 hours, or at least about 6.5 hours, or at least about 7.0 hours, or at least about 7.5 hours, or at least about 8.0 hours, or at least about 8.5 hours, or at least about 9.0 hours, or at least about 9.5 hours, or at least about 10 hours, or at least about 15 hours, or at least about 20 hours. In some aspects, the luminogenic caspase-3/7 substrate comprises a tetrapeptide sequence DEVD that is cleaved by caspase-3 or caspase-7, thereby producing a luciferase substrate. In some aspects, the luminescence-based assay is a Caspase-Glo® 3/7 assay system. In some aspects, the first plurality of cells, the second plurality of cells, the third plurality of cells or any combination thereof can be incubated with at least about 120 μl of Caspase-Glo® 3/7 assay reagent. The amount of Caspase-Glo® 3/7 assay reagent can be adjusted based on the microtiter plate this is being used for the assay. In a non-limiting example, when a 96 well microtiter plate is used, about 120 μl of Caspase-Glo® 3/7 assay reagent can be used.

In some aspects of the methods of the present disclosure, the first plurality of cells, the second plurality of cells, the third plurality of cells or any combination thereof can be incubated in a standard assay plate. This includes, but is not limited to, a microtiter, microplates, or microwell plates with 6, 12, 14, 48, 96, 384 or 1536 sample wells. The surface of the assay plate can be coated with a molecule to facilitate the attachment of cells onto the assay plate. Exemplary coatings include, but are not limited to, Poly-D-Lysine or Human Fibronectin. The assay plate can be left uncoated.

Any of the above aspects and embodiments can be combined with any other aspect or embodiment as disclosed here in the Summary and/or Detailed Description sections.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless defined otherwise, all technical and scientific terms used herein have the same meaning as commonly understood by one of ordinary skill in the art to which the invention pertains. Although other probes, compositions, methods, and kits similar, or equivalent, to those described herein can be used in the practice of the present disclosure, exemplary materials and methods are described herein. It is to be understood that the terminology used herein is for the purpose of describing particular aspects only, and is not intended to be limiting.

### EXAMPLES

In the following examples, Human Retinoblastoma (hRB) Y79 (ATCC® HTB-18™) cells were expanded and maintained for use by culturing in ATCC®-formulated RPMI-1640 medium supplemented with 20% fetal bovine serum. The cells were maintained according to ATCC® recommendations. Human retinal progenitor cells (hRPCs) and human retinal pigment epithelium cells (hRPE cells) were isolated from 18-20 week human fetuses and propagated in standard medium (SM) comprising Advanced DMEM/F12 with 1×N−2 supplement, 1×GlutaMax-1, 20 ng/ml human FGF-basic protein and 20 ng/ml human EGF protein.

In the following experiments, conditioned medium (CM) was collected from hRPCs, hRB cells or hRPE grown in SM for 48 hours (with replacement of the SM after 24 hours). Cells were harvested and counted after each CM collection.

### Example 1—Coated and Non-Coated Substrates

Different coating reagents were tested to determine a substrate suitable for use in the methods of the present disclosure.

Opaque 96 well assay plates were either left uncoated, coated with Poly-D-Lysine or coated with Human Fibronectin. The plates were coated with Poly-D-Lysine by incubating 25 μl to 50 μl of 200 μg/ml Poly-D-Lysine solution in each well for five minutes to two hours at room temperature. Alternatively, 250 μl of 200 μg/ml Poly-D-Lysine solution was incubated in each well for one hour at room temperature. After incubation, the plates were rinsed with ddH₂O, then left to air dry for two hours. The plates were coated with Human Fibronectin by incubating 300 μl of 20 μg/ml Human Fibronectin solution at 37° C. overnight. After overnight incubation, the plates were rinsed with Advanced DMEM/F12.

Human Retinoblastoma (hRB) Y79 (ATCC® HTB-18™) cells were then added to the three different assay plates. In the case of the Human Fibronectin plates, the hRB cells were observed to colonize. Conversely, in the Poly-D-Lysine plates, the hRB cells were observed to distribute evenly. When the cells were added to uncoated plates in human retinal progenitor cell standard medium, the cells attached to the plate despite the lack of coating. Thus, uncoated plates can also be used in the methods of the present disclosure.

### Example 2—a Fluorescence-Based Cell Viability Assay

A fluorescence-based assay to measure cell viability was tested for use in the methods of the present disclosure.

First, 12.5×10³, 2.5×10⁴, 5.0×10⁴, 1.0×10⁵ and 2.0×10⁵ Human Retinoblastoma (hRB) Y79 (ATCC® HTB-18™) cells were plated in five separate wells of a 96 well assay plate and incubated in 50 μl SM. After allowing the cells to settle for 30 minutes to two hours, the cells were then incubated with 250 μl of the human retinal progenitor cell conditioned medium after being mixed thoroughly. 200 μl of the media was then aspirated and 20 μl of diluted CellTiter-Blue® reagent (1:4 in DPBS) comprising resazurin (7-Hydroxy-3H-phenoxazin-3-one 10-oxide sodium salt) was added to the cells for 1, 2, 3 or 4 hours at 37° C. Resazurin is a compound that, when internalized by viable cells, is reduced to the compound known as resorufin. Resorufin is highly fluorescent, meaning that the fluorescence signal generated can be used to determine the viability of the cells incubated with Resazurin. Non-viable cells that lack metabolic capacity do not reduce resazurin to resorufin, and, thus, no fluorescence signal is generated.

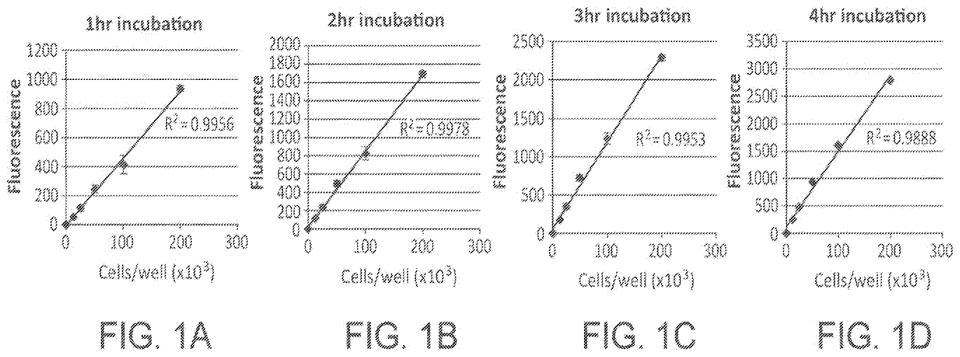

The fluorescence of each well was measured after incubation with resazurin. Fluorescence signals were read at an excitation wavelength of 520 nm and an emission wavelength of 580-640 nm. Each treatment was done in triplicates or quadruplicates. As shown in FIG. 1, there was a linear response (R²=0.99) for incubation times of 1, 2 or 3 hours for across all cell amounts. These results indicate that in order to achieve maximum treatment effect per cell and minimize experiment duration and intra-sample variation, the methods of the present disclosure can use 2.5×10⁴ cells/well and a 2 hour Resazurin incubation time.

### Example 3—Determining the Potency of Human Retinal Progenitor Cell Conditioned Media

The methods of the present disclosure were used to test the potency of human retinal progenitor cell conditioned media.

First, hRB cells were plated on an assay plate at a density of 2.5×10⁴ cells/well. The cells were then incubated with either human retinal progenitor cell conditioned medium (hRPC CM) or standard medium (SM; standard human retinal progenitor cell medium that has not been exposed to human retinal progenitor cells). The media was supplemented with no Sodium Butyrate, 2 mM Sodium Butyrate, 4 mM Sodium Butyrate or 8 mM Sodium Butyrate. Freshly made 200 mM Sodium Butyrate was added to 250 μl of media to generate the various sodium butyrate concentrations. The hRPC CM was produced using 9.0×10⁶ human retinal progenitor cells.

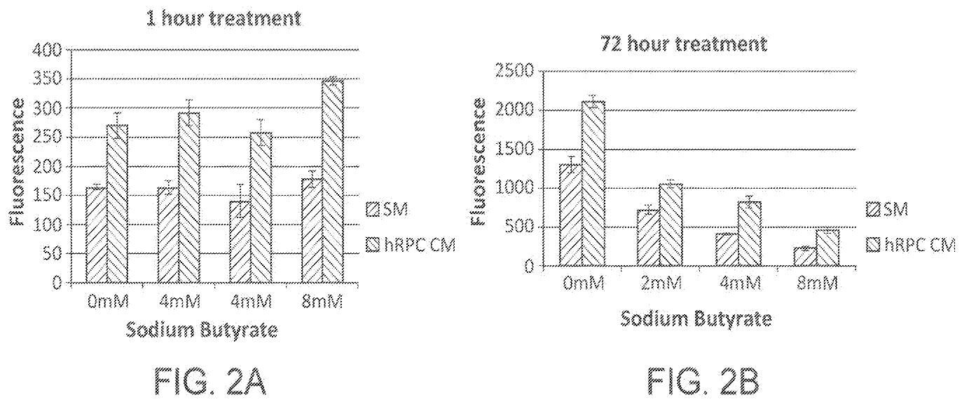

The hRB cells were incubated for either one hour or 72 hours in 300 μl of the various media. After incubation, cell viability was measured using the CellTiter-Blue® reagent as described above. Briefly, 200 μl of cell culture was mixed with 20 μl of diluted CellTiter-Blue® reagent and incubated for one to two hours. The fluorescence of each sample was then measured. As shown in FIG. 2, stronger fluorescence signals were observed after the 72 hour incubation (right panel) as compared to the one hour incubation (left panel) due to cell proliferation. However, significant dose dependent, sodium butyrate induced apoptosis was observed in the samples incubated for 72 hours, as higher concentrations of sodium butyrate resulted in decreased fluorescence signals, indicating decreased cell proliferation and viability.

Cells that were incubated with hRPC CM displayed increased fluorescence signals for both the one hour and 72 hour incubations as compared to the cells incubated with SM. These results indicate that the hRPC CM potently increased viability of the hRB cells even in the presence of the apoptosis-inducing sodium butyrate. The highest hRPC CM potency was observed in cells incubated with 8 mM sodium butyrate, in which the hRPC CM displayed about 2 fold potency in increasing cell viability compared to the SM at both the one hour and the 72 hour time points. These preliminary results indicated that 8 mM sodium butyrate provides the largest detectable signal.

In another experiment, hRB cells were plated on an assay plate at a density of 2.5×10⁴ cells/well. The cells were then incubated with either hRPC CM or SM. The media was supplemented with no sodium butyrate, 8 mM sodium butyrate, 16 mM sodium butyrate or 24 mM sodium butyrate. The hRPC CM was produced using 9.0×10⁶ human retinal progenitor cells.

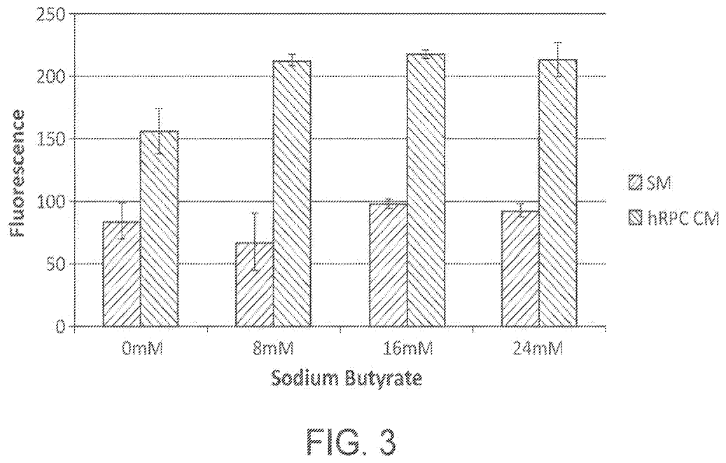

The hRB cells were incubated for one hour. After incubation, cell viability was measured using the CellTiterBlue® reagent, as described above. As shown in FIG. 3, hRB cells incubated with hRPC CM displayed increased fluorescence signals compared to the hRB cells incubated with SM. This trend was consistent across all four sodium butyrate concentrations. These results indicate that the hRPC CM potently increased viability of the hRB cells, even in the presence of a high concentration of apoptosis-inducing sodium butyrate.

### Example 4—Determining Potency of Human Retinal Progenitor Cell Conditioned Media at Different Doses

The methods of the present disclosure were used to test whether the potency of human retinal progenitor cell conditioned media was dose dependent.

First, hRB cells were plated on an assay plate at a density of 2.5×10⁴ cells/well. The cells were then incubated with hRPC CM, SM, hRPC CM diluted 2-fold with SM (0.5×hRPC CM), hRPC CM diluted 4-fold with SM (0.25×hRPC CM) or hRPC CM diluted 8-fold with SM (0.125×hRPC CM). The media was supplemented with either no sodium butyrate or 8 mM sodium butyrate. The hRPC CM was produced using 9.0×10⁶ human retinal progenitor cells.

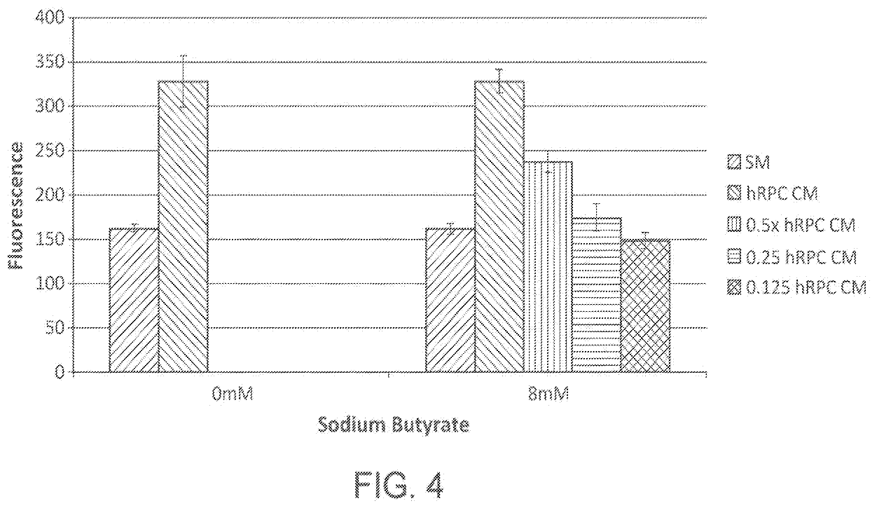

The hRB cells were incubated for one hour. After incubation, cell viability was measured using the CellTiterBlue® reagent, as described above. As shown in FIG. 4, the cells incubated with more concentrated hRPC CM displayed increased fluorescence signals compared to the cells incubated with more dilute hRPC CM and cells incubated with SM. These results indicate that hRPC CM potently increased viability of the hRB cells in a dose dependent manner.

In another experiment, hRB cells were plated on an assay plate at a density of 2.5×10⁴ cells/well. The cells were then incubated with hRPC CM produced using 9.0×10⁶ human retinal progenitor cells (hRPC CM 9M), hRPC CM produced using 6.0×10⁶ human retinal progenitor cells (hRPC CM 6M) or SM. The media was supplemented with either no sodium butyrate or 8 mM sodium butyrate.

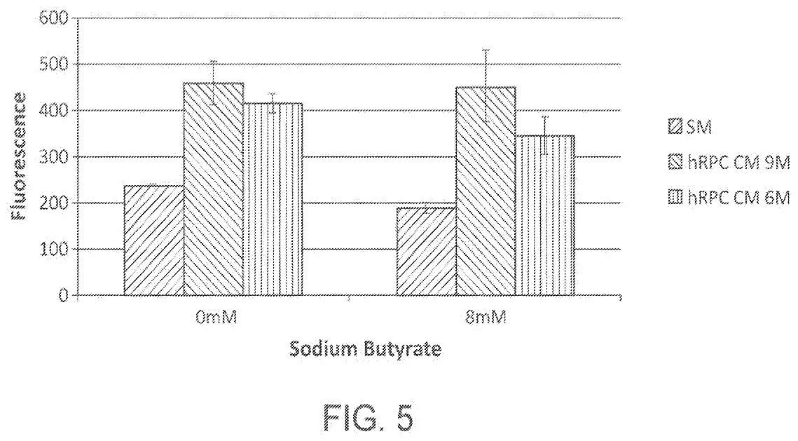

The hRB cells were incubated for one hour. After incubation, cell viability was measured using the CellTiterBlue® reagent, as described above. As shown in FIG. 5, the cells incubated with hRPC CM 9M displayed increased fluorescence values compared to the cells incubated with hRPC CM 6M and cells incubated with SM at both sodium butyrate concentrations. This result indicates that the potency of hRPC CM treatment is dependent upon the number of hRPCs used to produce the conditioned media.

These results indicate that the methods as provided herein can be used to determine the potency of hRPC-base therapy for the treatment of retinitis pigmentosa.

### Example 5—Determining if the Potency of Human Retinal Progenitor Cell Conditioned Media is Reproducible Using Different hRPC Populations

The methods of the present disclosure were used to test whether the potency of human retinal progenitor cell conditioned media was reproducible when the conditioned media was produced using separate populations of hRPCs from different product lots.

First, hRB cells were plated on an assay plate at a density of 2.5×10⁴ cells/well. The cells were then incubated with hRPC CM produced using 5.4×10⁶/10 ml hRPCs from a lot designated G1 (hRPC CM G1 5.4), hRPC CM produced using 3.7×10⁶/10 ml hRPCs from a lot designated G2 (hRPC CM G2 3.7), hRPC CM produced using 4.5×10⁶/10 ml hRPCs from a lot designated G3 (hRPC CM G3 4.5), hRPC CM produced using 5.0×10⁶/10 ml hRPCs from a lot designated G5 (hRPC CM G5 5.0) or SM. The media was supplemented with either no sodium butyrate or 8 mM sodium butyrate.

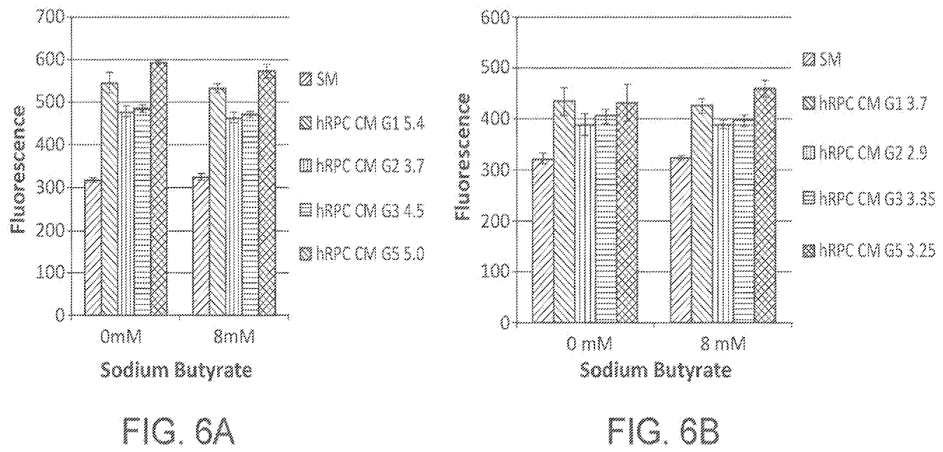

The hRB cells were incubated for 2 hours. After incubation, cell viability was measured using the CellTiterBlue® reagent, as described above. As shown in the left panel of FIG. 6, the cells incubated with hRPC CM displayed increased fluorescence values compared to the cells incubated with SM at both sodium butyrate concentrations. This increase was observed regardless of the lot of hRPCs used to generate the conditioned medium.

In another experiment, hRB cells were plated on an assay plate at a density of 2.5×10⁴ cells/well. The cells were then incubated with hRPC CM produced using 3.7×10⁶/10 ml hRPCs from a lot designated G1 (hRPC CM G1 3.7), hRPC CM produced using 2.9×10⁶/10 ml hRPCs from a lot designated G2 (hRPC CM G2 2.9), hRPC CM produced using 3.35×10⁶/10 ml hRPCs from a lot designated G3 (hRPC CM G4 3.35), hRPC CM produced using 3.25×10⁶/10 ml hRPCs from a lot designated G5 (hRPC CM G5 3.25) or SM. The media was supplement with either no sodium butyrate or 8 mM sodium butyrate.

The hRB cells were incubated for a 2 hours. After incubation, cell viability was measured using the CellTiterBlue® reagent, as described above. As shown in the right panel of FIG. 6, the cells incubated with hRPC CM displayed increased fluorescence values compared to the cells incubated with SM at both sodium butyrate concentrations. This increase was observed regardless of the source of the hRPCs used to generate the conditioned media.

In another experiment, hRB cells were plated on an assay plate at a density of 2.5×10⁴ cells/well. The cells were then incubated with hRPC CM produced using 2.038×106/4 ml hRPCs from a lot designated G1 (hRPC CM G1 2.038), hRPC CM produced using 1.774×106/4 ml hRPCs from a lot designated G2 (hRPC CM G2 1.774), hRPC CM produced using 1.543×106/4 ml hRPCs from a lot designated G3 (hRPC CM G3 1.543), hRPC CM produced using 1.542×106/4 ml hRPCs from a lot designated G4 (hRPC CM G4 1.542), hRPC CM produced using 1.318×106/4 ml hRPCs from a lot designated G5 (hRPC CM G5 1.318), hRPC CM produced using 1.977×106/4 ml hRPCs from a CMO Seed Bank Sample designated L-SB (hRPC CM L-SB), hRPC CM produced using 1.162×106/4 ml hRPCs from a L Product Bank Sample designated L-PB (hRPC CM L-PB), or SM. The media was supplemented with either no sodium butyrate or 8 mM sodium butyrate.

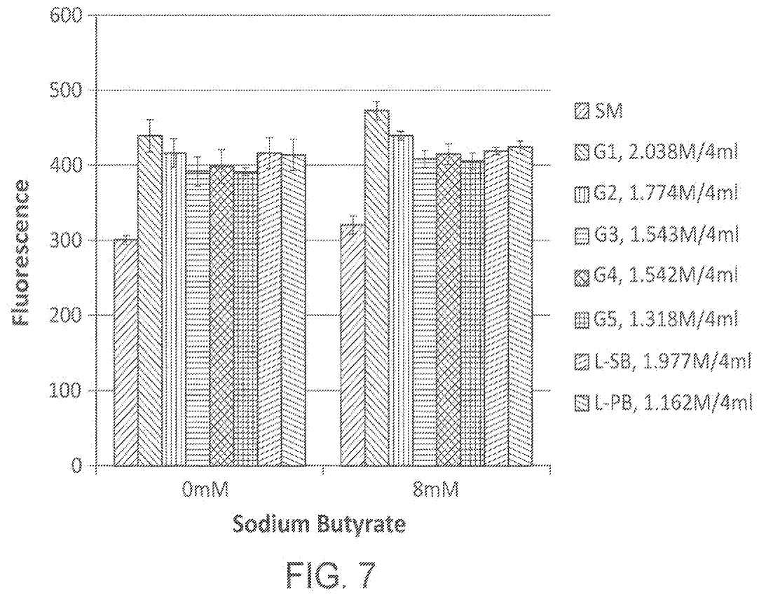

The hRB cells were incubated for 2 hours. After incubation, cell viability was measured using the CellTiterBlue® reagent, as described above. As shown in FIG. 7, the cells incubated with hRPC CM displayed increased fluorescence values compared to the cells incubated with SM at both sodium butyrate concentrations. This increase was observed regardless of the source of the hRPCs used to generate the conditioned media.

The results in this example indicate that the different populations of hRPCs can be used to produce potent conditioned media. The potency of conditioned medium from different hRPC lots showed consistently elevated metabolic activity versus the SM. While there was some differences in potency between lots, this is most likely due to the lack of standardization at the time of conditioned medium collection.

### Example 6—Determining if the Potency of Human Retinal Progenitor Cell Conditioned Media is Specific

The methods of the present disclosure were used to test whether the potency of hRPC CM was specific to conditioned media produced using hRPCs versus conditioned media produced using other cell types.

First, hRB cells were plated on an assay plate at a density of 2.5×10⁴ cells/well. The cells were then incubated with hRPC CM produced using 9.0×10⁶ human retinal progenitor cells (hRPC CM 9M), hRB conditioned media produced using 12.2×10⁶ hRB cells (hRB CM 12.2M) or SM. The media was supplemented with either no sodium butyrate or 8 mM sodium butyrate.

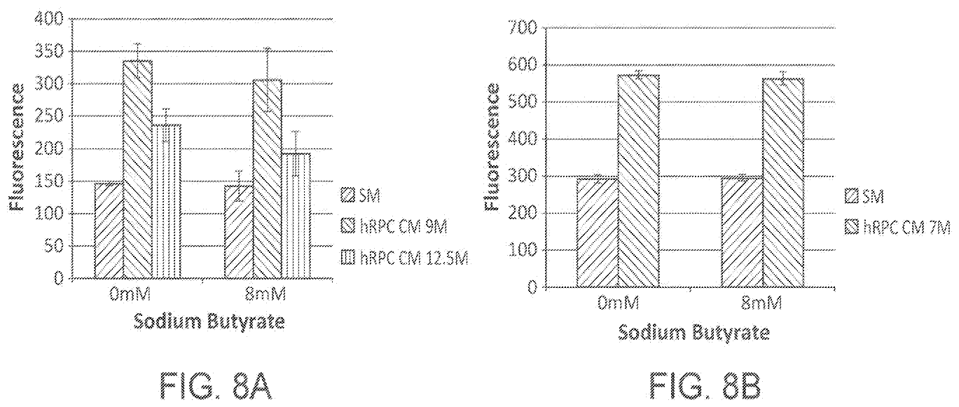

The hRB cells were incubated for 1 or 2 hours. After incubation, cell viability was measured using the CellTiterBlue® reagent, as described above. As shown in the left panel of FIG. 8, the cells incubated with hRPC conditioned media displayed increased fluorescence signals compared to cells incubated with hRB conditioned media and SM. This trend was consistent across sodium butyrate concentrations.

In another experiment, hRB cells were plated on an assay plate at a density of 2.5×10⁴ cells/well. The cells were then incubated with human retinal pigment epithelial cell conditioned media produced using 7.0×10⁶ human retinal pigment epithelial cells (hRPE CM 7M) or standard media (SM; standard human retinal progenitor cell media that has not been exposed to human retinal progenitor cells). The media was supplemented with either no sodium butyrate or 8 mM sodium butyrate.

The hRB cells were incubated for 1 or 2 hours. After incubation, cell viability was measured using the CellTiterBlue® reagent, as described above. As shown in FIG. 8, the cells incubated with hRPE conditioned media displayed increased fluorescence signals compared to the cells incubated with SM. This trend was consistent across sodium butyrate concentrations.

### Summary of Examples 1-6

While the target cell type used in the preceding examples (hRB line) differs substantially from the anticipated in vivo cell targets (i.e., patients' retina), the methods of the present disclosure nevertheless demonstrates the ability to detect and discriminate different levels of diffusible trophic activity from the therapeutic cells. The selection of a highly abnormal target cell for use in the assay makes the methods of the present disclosure highly adaptable to a variety of different disease contexts, particularly those that may have a target cell type that is recalcitrant to in vitro experimentation.

### Example 7—Conditioned Media Collection

The following example describes the production and collection of conditioned media for use in the methods of the present disclosure. More specifically, this example describes the collection of conditioned media used to grow hRPCs.

First vials containing hRPC cells were removed from liquid nitrogen and their caps were loosened in a cell culture hood to release pressure. The caps were then re-tightened. The hRPC cells were then thawed by placing the vials at 37° C. water bath for 2-3 minutes until ice crystals disappear. The entire cell suspension (˜1 ml/vial) was then transferred using a 1 ml pipette tip into a 15 ml conical bottom tube. The vial was rinsed with fresh cold standard media (SM) 1-2 times and the SM used to rinse was added to the cell suspension dropwise. The entire cell mixture was then shaken gently.

10-14 ml of cold fresh SM was added to the cell suspension in the 15 ml conical bottom tube and the mixture was shaken gently. The cells were then pelleted by centrifuging the tube at 300×g for 5 minutes. The supernatant was then aspirated and discarded. Fresh cold SM was then used to resuspend the cell pellet using a 1 ml pipette tip to gently pipette the cell pellet up and down 6-8 times. The cell viability and cell number were then measured using a Hemocytometer or Countess Counter. Based on the measured cell number, 8 million dissociated live cells were seeded into a new T75 flask pre-coated with fibronectin in 10 ml fresh SM. The cell culture was then gently rocked immediately.

After seeding, the cells were inspected by inverted microscope to ensure that the cells were evenly distributed in the cell culture flask. If uneven distribution was observed, the cells were gently rocked further until even distribution was achieved. The cells were then incubated at 37° C. and 5% CO₂.

The next day, the cells were checked under an inverted microscope and their status was recorded. Following observation, the entire cell culture media was aspirated from the hRPC culture flask and discarded. Then, 10 ml of pre-warmed (37° C.) SM was added to each T75 flask. The cells were then incubated at 37° C. and 5% CO₂.

The next, the status of the cells was checked again under an inverted microscope. The conditioned media was then aspirated and collected from the hRPC culture flasks. The media was then centrifuged for 5 minutes at 475×g. The centrifuged conditioned media was then placed on a cold block. The conditioned media was then aliquoted into 1.5 ml tubes with care given to avoid aspirating and of the pellet at the bottom of the tube containing dead cells and cell debris. The aliquoted conditioned media was then immediately transferred to −80° C. for long-term storage for use in the methods of the present disclosure.

Subsequently, 5 ml of pre-warmed 37° C. SM media was added to the T75 culture flasks containing the hRPC cells. The flasks were then gently rocked and the media was aspirated and discarded. 4 ml of TrypLE Select working solution was then added to each T75 flask, and the flask was then gently rocked to completely cover the surface on which the cells were growing with the TrypLE Select working solution. The cells were incubated in the TrypLE Select working solution for five minutes at 37° C. and 5% CO₂.

After the incubation, the cells were examined under an inverted microscope to ensure that at least 95% of cells were detached from the cell culture surface. If further detachment was necessary, the culture flask was further agitated.

To stop the enzymatic dissociation of cells, 5 ml of SM was added to the T75 flask. The media was then titrated gently to wash cells off the surface of the flask using a sterile serological pipette. The cell suspension was then transferred to a sterile 15 ml tube. The tube was then placed in a cold block. The T75 flask was then rinsed again using an additional 5 ml of fresh SM, which was then transferred to the sterile 15 ml tube. The 15 ml conical tube containing the cells were then centrifuged in a tabletop centrifuge at 300×g for 5 minutes at 4° C. After centrifugation, the supernatant was aspirated and discarded. Cold (4° C.) BSS Plus solution was then added to the pelleted cells. The pellet was then resuspend using a lml pipette tip to gently pipette the BSS Plus solution up and down 6-10 times. The suspension was continuously kept cold. Finally, the cell viability and cell number was measured using a Hemocytometer or Countess Counter.

### Example 8—Multiplexed Potency Assay of the Present Disclosure

This example describes various experiments comprising the multiplexed potency assay of the present disclosure and its use in determining the potency of a cell-based therapy or treatment.

In a non-limiting example of a multiplexed potency assay, RB cells were plated into a 96-well plate. For each well, 25,000 RB cells were plated in 25 μl of SM. Each well was then treated with either 75 μl of conditioned media (in some cases, diluted conditioned media depending on the experiment) or 75 μl of a control media (standard media, a positive-control conditioned media, a negative-control conditioned media). Sodium butyrate was then added to each well to a final concentration of 16 mM. In some experiments, sodium butyrate was added to a final concentration of 8 mM, 16 mM or 32 mM, or no sodium butyrate was added at all, to determine the effect of sodium butyrate concentration on the output of the assay. The cells were then incubated for 46 hours at 37° C.

After 46 hours of incubation, 20 μl of CellTiter-Blue® reagent (diluted 1:4 with Dulbecco's PBS) was added to each well. The cells were then incubated at 37° C. After incubation, the viability of the cells was measured by recording fluorescence of each well at (530_{Ex}/590_{Em}).

After measuring cell viability, an equal volume (120 μl) of Caspase-Glo® 3/7 reagent was added to each well. The cells were then incubated at room temperature for 1.5 hours to achieve steady state of the luciferase output. After incubation, the luminescence of each well was measured to determine apoptosis activity in each well. The Fold Change Protection value for each well was then calculate as described above. A potency value for each well was then calculated by taking the ratio of the Fold Change Protection value of each well and normalizing to the Fold Change Protection Value calculated for the well using a control media, more specifically standard media.

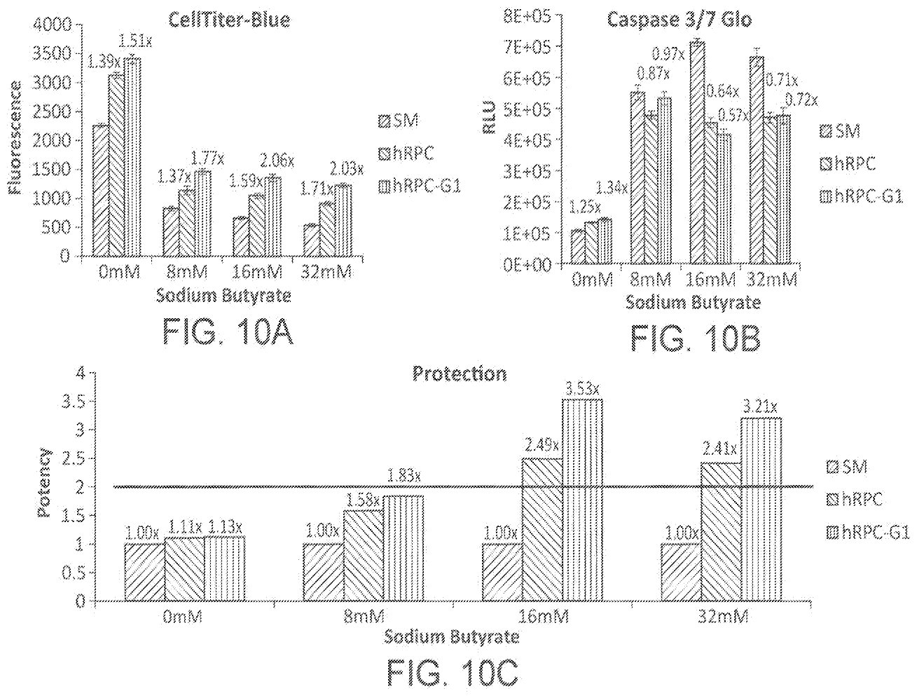

FIG. 10 shows the results from an experiment using the preceding multiplexed method with varying amounts of sodium butyrate as well as conditions comprising standard media, conditioned media from one population of hRPCs or conditioned media from a different population of hRPCs. The top left panel of FIG. 10 shows the measured viability for each condition using the CellTiter-Blue reagent. The top right panel of FIG. 10 shows the measured apoptosis activity for each condition using the Caspase-Glo® 3/7 reagent. The bottom panel of FIG. 10 shows the potency value, as calculated described above. These results indicate that the conditioned media from both populations of hRPCs protect the RB cells from the deleterious effects of the sodium butyrate. Additionally, the results indicate that the use 16 mM sodium butyrate results in the largest potency signals using the multiplexed potency assay of the present disclosure.

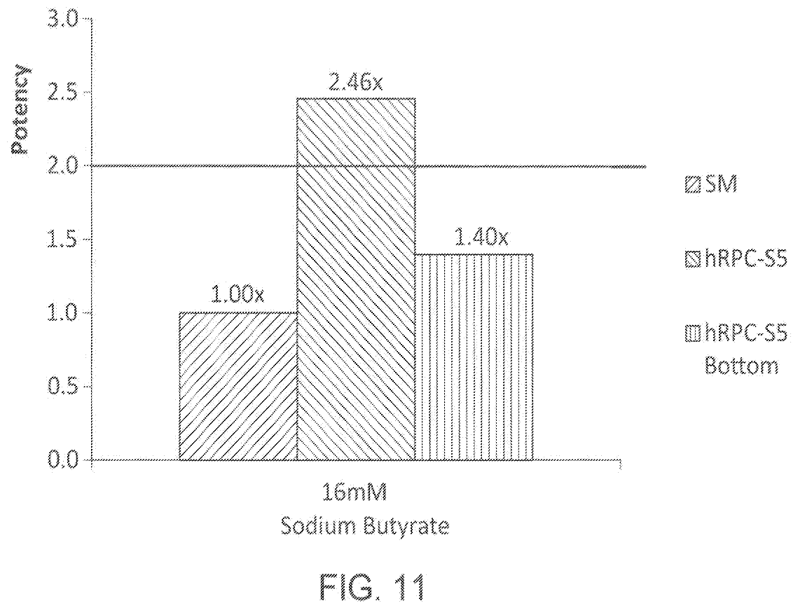

FIG. 11 shows the results from an experiment using the preceding multiplexed method using 16 mM sodium butyrate and conditioned media that was either unfiltered or filtered. Briefly, 10 ml of conditioned media from hRPCs was added to an Amicon® Ultra 3K filter device (Millipore UFC900324). Using a swinging-bucket rotor, the device with the conditioned media was centrifuged at 4,000×g for about 10-15 minutes. The “top” or “retentate” fraction was recovered by inserting a pipette into the filter device and withdrawing the sample with a side-to-side sweeping motion to ensure total recovery. The “bottom” or “filtrate” fraction was recovered by removing the filter device and collecting the part of the sample that flowed through the filter. As shown in FIG. 11, the unfiltered conditioned media protected the RB cells from the deleterious effects of the sodium butyrate. In contrast, the “bottom” or “filtrate” fraction did not protect the RB cells to the same extent.

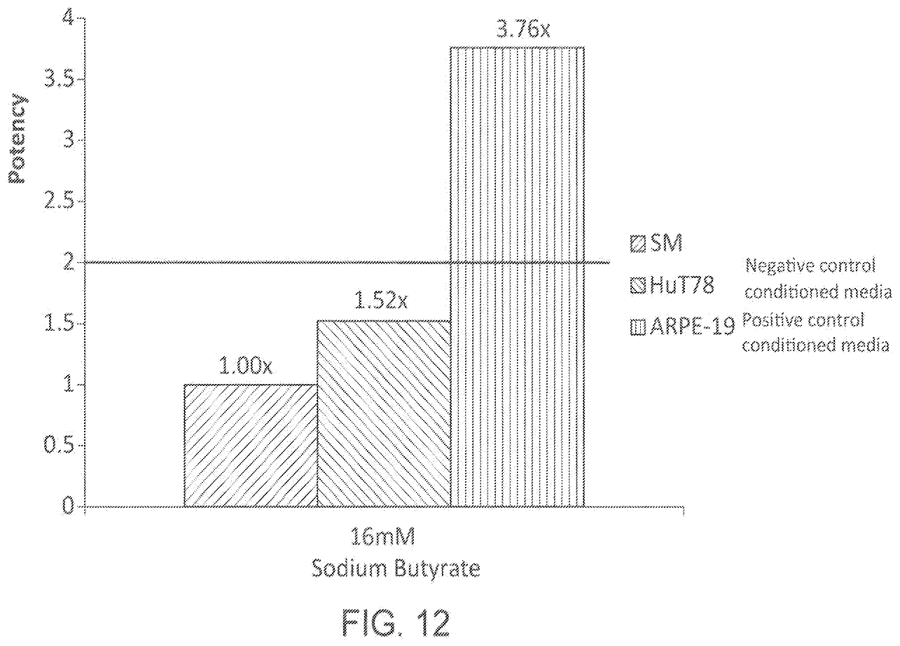

FIG. 12 shows the results from an experiment using the preceding multiplexed method using 16 mM sodium butyrate as well as standard media, conditioned media from HuT78 cells and conditioned media from ARPE-19 cells. The conditioned media from the HuT78 cells is a negative control conditioned media, as this conditioned media is expected not to protect the RB cells from the deleterious effects of sodium butyrate. The conditioned media form the ARPE-19 cells is a positive control conditioned media, as this conditioned media is expected to protect the RB cells from the deleterious effects of sodium butyrate. As shown in FIG. 12, while the ARPE-19 conditioned media protected the RB cells, as expected, the HuT78 conditioned media did not. Thus, the methods of the present disclosure can comprise the use of positive and negative control conditioned media to ensure the integrity of the assay being performed.

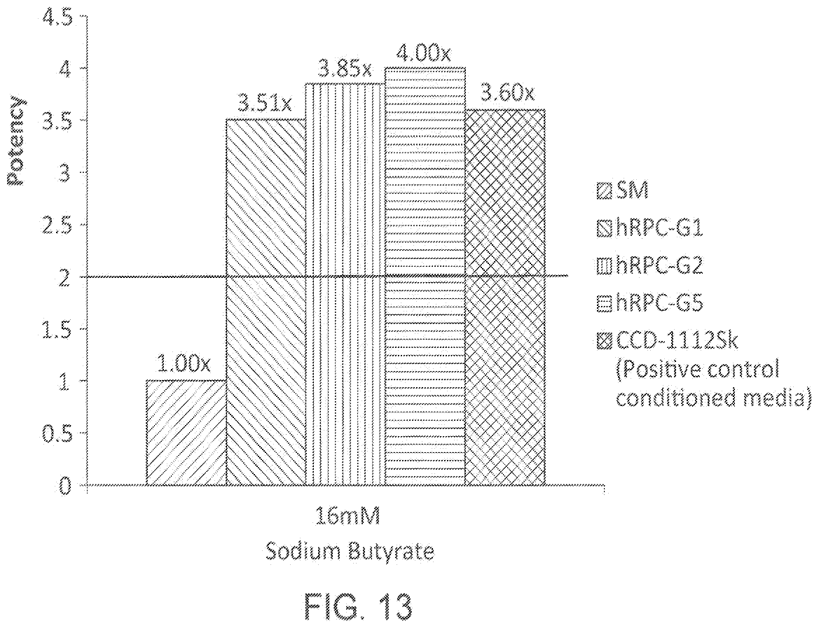

FIG. 13 shows the results from an experiment using the preceding multiplexed method using 16 mM sodium butyrate as well as standard media, conditioned media from three different “lots” (different populations, G1, G2 and G5) of hRPCs and conditioned media from CCD-1112Sk cells. The conditioned media from the CCD-1112Sk cells is a positive control conditioned media, as this conditioned media is expected to protect the RB cells from the deleterious effects of sodium butyrate. As shown in FIG. 13, the conditioned media from all three lots of hRPCs and the positive control conditioned media from the CCD-1112Sk cells all protected the RB cells from the deleterious effects of sodium butyrate. Thus, these results indicate that the methods of the present disclosure can be used to test independent “lots” of different cell-based treatments or therapies and that the measured potency values are robust.

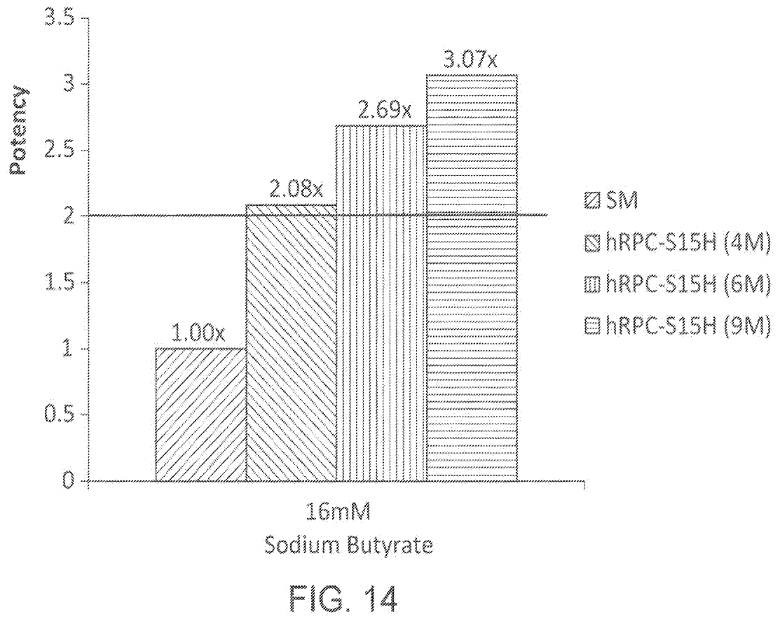

FIG. 14 shows the results from an experiment using the preceding multiplex method using 16 mM sodium butyrate as well as standard media and conditioned media from cultures of hRPCs with varying seeding densities. The conditioned media was collected using a method similar to that in Example 7, except that seeding densities of 4 million, 6 million or 9 million cells were used. As shown in FIG. 14, conditioned media derived from hRPC cultures with higher seeding density provide more protection to the RB cells from the deleterious effects of sodium butyrate. Thus, the measured potency values of the methods of the present disclosure can exhibit dose dependency.

### Example 9—the Methods of the Present Disclosure Exhibit Linearity

This example describes an experiment that demonstrates the methods of the present disclosure exhibit linearity in measured potencies when a dilution series of conditioned media (CM) is used.

First, RB cells were plated into a 96-well plate. For each well, 25,000 RB cells were plated in 25 μl of SM. A dilution series of conditioned media (for example produced using the methods of Example 7) was then prepared as follows:
i. 100% CM: CM only
ii. 75% CM: 75% CM+25% SM
iii. 50% CM: 50% CM+50% SM
iv. 25% CM: 25% CM+75% SM
v. 12.5% CM: 12.5% CM+87.5% SM
vi. 0% CM: 100% SM

75 μl of each of the conditioned media (a-f) was added to separate wells in the 96-well plate. Sodium butyrate was then added to each well for a final concentration of 16 mM sodium butyrate. The cells were then incubated for 46 hours at 37° C.

After 46 hours of incubation, 20 μl of CellTiter-Blue® reagent (diluted 1:4 with Dulbecco's PBS) was added to each well. The cells were then incubated at 37° C. After incubation, the viability of the cells was measured by recording fluorescence of each well at (530_{Ex}/590_{Em}).

After measuring cell viability, an equal volume (120 μl) of Caspase-Glo® 3/7 reagent was added to each well. The cells were then incubated at room temperature for 1.5 hours to achieve steady state of the luciferase output. After incubation, the luminescence of each well was measured to determine apoptosis activity in each well.

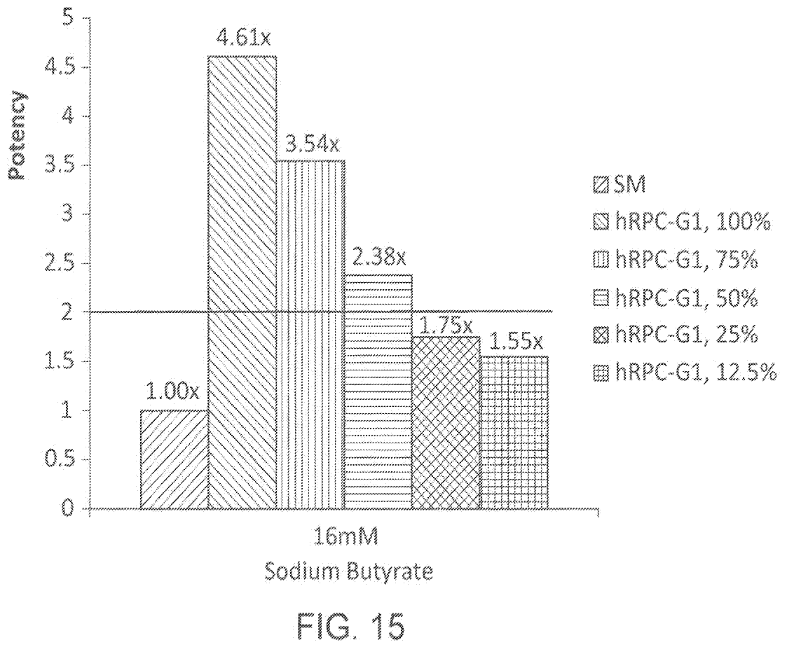

The Fold Change Protection value for each well was then calculate as described above. A potency value for each well was then calculated by taking the ratio of the Fold Change Protection value of each well and normalizing to the Fold Change Protection Value calculated for the well comprising 0% CM (100% SM). As shown in FIG. 15, the output of the method exhibits linearity. The condition comprising 100% CM had a measured potency value of approximately 4.61. The condition comprising 75% CM had a measured potency value of approximately 3.54, which is approximately the same as 0.75× the potency value of the 100% CM condition (0.75×4.61). Similarly, the condition comprising 50% CM had a measured potency value of approximately 2.38, which is approximately the same as 0.5× the potency value of the 100% CM condition (0.5×4.61). Thus, the methods of the present disclosure provide a potency output that is linear.

### EQUIVALENTS

The details of one or more embodiments as provided herein are set forth in the accompanying description above. Although any methods and materials similar or equivalent to those described herein can be used in the practice or testing of the present invention, exemplary methods and materials are now described.

The foregoing description has been presented only for the purposes of illustration and is not intended to limit the invention to the precise form disclosed, but by the claims appended hereto.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

1. A method for measuring the potency of a cell-based therapy or treatment, the method comprising the steps of:
incubating a first plurality of cells with a toxic compound and conditioned media, wherein the conditioned media comprises the media used to culture the cell-based therapy or treatment;
incubating an at least second plurality of cells with the toxic compound and control media;
determining the viability of the first plurality of cells and the at least second plurality of cells; and
comparing the viability of the first plurality of cells with the viability of the second plurality of cells, thereby determining the potency of the cell-based therapy or treatment.

2. The method of claim 1, wherein the potency is the ratio of the viability of the first plurality of cells with the viability of the second plurality of cells.

3. A method for measuring the potency of a cell-based therapy or treatment, the method comprising the steps of:
incubating a first plurality of cells with a toxic compound and conditioned media; wherein the conditioned media comprises the media used to culture the cell-based therapy or treatment;
incubating an at least second plurality of cells with the toxic compound and control media;
determining the viability of the first plurality of cells and the at least second plurality of cells;
determining the apoptosis activity in the first plurality of cells and the at least second plurality of cells;
determining a fold change protection value of the first plurality of cells, wherein the fold change protection value is the ratio of viability of the first plurality of cells to the apoptosis activity in the first plurality of cells;
determining a fold change protection value of the at least second plurality of cells; wherein the fold change protection value is the ratio of viability of the at least second plurality of cells to the apoptosis activity in the at least second plurality of cells; and
determining the potency of the cell-based therapy or treatment, wherein the potency is the ratio of the fold change protection value of the first plurality of cells to the fold change protection value of the at least second plurality of cells.

4. The method of claim 1, further comprising:
comparing the potency of the cell-based therapy or treatment to a predetermined cutoff value,
wherein if the potency is greater than the predetermined cutoff value then the cell-based therapy or treatment is identified as sufficiently potent for administration to a subject.

5. The method of claim 1, further comprising:
comparing the potency of the cell-based therapy or treatment to a predetermined cutoff value; and
administering to a subject in need thereof at least one therapeutically effective dose of the cell therapy or treatment when the potency is greater than the predetermined cutoff value.

6. The method of claim 4, wherein the predetermined cutoff value is about 2.

7. The method of claim 1, wherein the cell-based therapy or treatment comprises retinal progenitor cells (RPCs), retinal pigment epithelial cells (RPEs), ARPE-19 cells, neural stem/progenitor cells, mesenchymal stem cells, CD34+ cells, stem/progenitor cells, leukocytes, fibroblasts, or any combination thereof.

8. The method of claim 1, wherein the cell-based therapy or treatment comprises exosomes derived from cells selected from retinal progenitor cells (RPCs), retinal pigment epithelial cells (RPEs), ARPE-19 cells, neural stem/progenitor cells, mesenchymal stem cells, CD34+ cells, stem/progenitor cells, leukocytes, fibroblasts, and any combination thereof.

9. The method of claim 1, wherein the cell-based therapy or treatment comprises RPCs.

10. The method of claim 1, wherein the first plurality of cells and the at least second plurality of cells comprise retinoblastoma (RB) cells, retinal pigment epithelial cells (RPEs), ARPE-19 cells, Muller cell-derived cells, MIO-M1 cells, neuronal cells, glial cells, fibroblasts, non-ocular cells, or any combination thereof.

11. The method of claim 1, wherein the first plurality of cells and the at least second plurality of cells comprise RB cells.

12. The method of claim 1, wherein the first plurality of cells and the at least second plurality of cells comprise at least about 1,000 RB cells to at least about 250,000 RB cells in at least about 10 ml to at least about 40 ml of media.

13. The method of claim 1, wherein the first plurality of cells and the at least second plurality of cells comprise at least about 25,000 RB cells in at least about 25 ml of media.

14. The method of claim 1, wherein the first plurality of cells and the at least second plurality of cells are incubated with at least about 50 ml to at least about 100 ml of conditioned media and control media, respectively.

15. The method of claim 1, wherein the first plurality of cells and the at least second plurality of cells are incubated with at least about 75 ml of conditioned media and control media, respectively.

16. The method of claim 1, wherein the toxic compound induces apoptosis.

17. The method of claim 1, wherein the toxic compound is sodium butyrate,
wherein optionally the sodium butyrate is present in a concentration of about 2 mM to about 32 mM,
and optionally the sodium butyrate is present in a concentration of about 16 mM.

18-19. (canceled)

20. The method of claim 1, wherein the first plurality of cells and the at least second plurality of cells are incubated for a time period of at least about 1 hour to at least about 72 hours.

21. The method of claim 1, wherein the first plurality of cells and the at least second plurality of cells are incubated for a time period of at least about 46 hours.

22. The method of claim 1, wherein determining the viability of the first plurality of cells and the at least second plurality of cells comprises measuring metabolic capacity of the first plurality of cells and the at least second plurality of cells.

23. The method of claim 22, wherein the metabolic capacity is measured using a fluorescence-based assay,
wherein optionally the fluorescence-based assay comprises: incubating the first plurality of cells and the at least second plurality of cells with resazurin (7-Hydroxy-3//-phenoxazin-3-one IO-oxide sodium salt) for at a period of at least about 1 hour; and measuring the fluorescence of the first plurality of cells and the at least second plurality of cells;
and optionally the fluorescence-based assay is a CellTiter-Blue® Cell Viability Assay,
and optionally at least about 20 ml of 1:4 diluted CellTiter-Blue® reagent is added to the first plurality of cells and to the at least second plurality of cells.

24-26. (canceled)

27. The method of claim 3, wherein the apoptosis activity in the first plurality of cells and the at least second plurality of cells is measured using a luminescence-based assay,
wherein optionally the luminescence-based assay comprises: incubating the first plurality of cells and the at least second plurality of cells with a luminogenic caspase-3/7 substrate for at least about 1 hours; and measuring the luminescence of the first plurality of cells and the at least second plurality of cells,
and optionally the luminogenic caspase-3/7 substrate comprises a tetrapeptide sequence DEVD that is cleaved by caspase-3 or caspase-7, thereby producing a luciferase substrate,
and optionally the luminescence-based assay is a Caspase-Glo® 3/7 assay system,
and optionally at least about 120 ml of Caspase-Glo® 3/7 assay reagent is added to the first plurality of cells and to the at least second plurality of cells.

28-31. (canceled)

32. The method of claim 31, further comprising:
incubating an at least third plurality of cells with a toxic compound and inactive conditioned media,
wherein the inactive conditioned media comprises the media used to culture an inactive cell-based therapy or treatment;
determining the viability of the at least third plurality of cells;
determining the apoptosis activity in the at least third plurality of cells;
determining a fold change protection value of the at least third plurality of cells, wherein the fold change protection value is the ratio of viability to apoptosis activity;
determining the potency of the inactive cell-based therapy or treatment, wherein the potency is the ratio of the fold change protection value of the at least third plurality of cells to the fold change protection value of the at least second plurality of cells; and
comparing the potency of the inactive cell-based therapy or treatment to a predetermined cutoff value, wherein if the potency of the inactive cell-based therapy is less than or equal to the predetermined cutoff value, then the method is identified as valid,
wherein optionally the inactive cell-based therapy or treatment comprises cutaneous T lymphocytes, HuT 78 cells or any combination thereof.

33. The method of claim 1, further comprising:
incubating an at least third plurality of cells with a toxic compound and inactive conditioned media, wherein the inactive conditioned media comprises the media used to culture an inactive cell-based therapy or treatment;
determining the viability of the at least third plurality of cells;
comparing the viability of the third plurality of cells with the viability of the second plurality of cells, thereby determining the potency of the inactive cell-based therapy or treatment; and
comparing the potency of the inactive cell-based therapy or treatment to a predetermined cutoff value, wherein if the potency of the inactive cell-based therapy is less than or equal to the predetermined cutoff value, then the method is identified as valid,
wherein optionally the inactive cell-based therapy or treatment comprises cutaneous T lymphocytes, HuT 78 cells or any combination thereof.

34. (canceled)

35. The method of claim 3, further comprising:
incubating an at least third plurality of cells with a toxic compound and active conditioned media, wherein the active conditioned media comprises the media used to culture an active cell-based therapy or treatment;
determining the viability of the at least third plurality of cells;
determining the apoptosis activity in the at least third plurality of cells;
determining a fold change protection value of the at least third plurality of cells,
wherein the fold change protection value is the ratio of viability to apoptosis activity;
determining the potency of the active cell-based therapy or treatment, wherein the potency is the ratio of the fold change protection value of the at least third plurality of cells to the fold change protection value of the at least second plurality of cells; and the potency of the active cell-based therapy or treatment to a predetermined cutoff value,
wherein if the potency of the active cell-based therapy is greater than the predetermined cutoff value, then the method is identified as valid,
wherein optionally the active cell-based therapy comprises retinal pigment epithelial cells (RPEs), ARPE-19 cells, fibroblasts, CCD-11 I2Sk cells or any combination thereof.

36. The method of claim 1, further comprising:
incubating an at least third plurality of cells with a toxic compound and active conditioned media, wherein the active conditioned media comprises the media used to culture an active cell-based therapy or treatment;
determining the viability of the at least third plurality of cells;
comparing the viability of the third plurality of cells with the viability of the second plurality of cells, thereby determining the potency of the active cell-based therapy or treatment; and
comparing the potency of the active cell-based therapy or treatment to a predetermined cutoff value, wherein if the potency of the active cell-based therapy is greater than the predetermined cutoff value, then the method is identified as valid,
wherein optionally the active cell-based therapy comprises retinal pigment epithelial cells (RPEs), ARPE-19 cells, fibroblasts, CCD-11 I2Sk cells or any combination thereof.

37. (canceled)

38. The method of claim 1, wherein the control media comprises standard media.

39. The method of claim 1, wherein the cell-based therapy or treatment is for treating a retinal disease or condition.
